(19) United States
(12) Patent Application Publication
(10) Pub. No.: US 2017/0058282 A1
(43) Pub. Date: Mar. 2, 2017

(54) GENETICALLY ENGINEERED SENSORS FOR IN VIVO DETECTION OF BLEEDING

(71) Applicant: Massachusetts Institute of Technology, Cambridge, MA (US)

(72) Inventors: Timothy Kuan-Ta Lu, Cambridge, MA (US); Mark K. Mimee, Cambridge, MA (US)

(73) Assignee: Massachusetts Institute of Technology, Cambridge, MA (US)

(21) Appl. No.: 15/205,625

(22) Filed: Jul. 8, 2016

Related U.S. Application Data
(60) Provisional application No. 62/190,709, filed on Jul. 9, 2015.

(51) Int. Cl. C12N 15/70; C12Q 1/02; C12Q 1/68; C12N 15/63; A61K 49/00

(52) U.S. Cl. CPC C12N 15/70; C12N 15/635; A61K 49/0017; C12Q 1/6897; C12Q 1/02

(57) ABSTRACT

Provided herein are microorganisms engineered with heme-responsive transcription factors and genetic circuits. Also provided are methods for using engineered microorganisms to sense bleeding events and treat bleeding in vivo.

### RELATED APPLICATIONS

This application claims the benefit under 35 U.S.C. §119(e) of U.S. provisional application No. 62/190,709, filed Jul. 9, 2015, which is incorporated by reference herein in its entirety.

### FEDERALLY SPONSORED RESEARCH

The invention was made with Government support under Contract No. FA8721-05-C-0002 awarded by the U.S. Air Force. The Government has certain rights in the invention.

### FIELD OF INVENTION

The present disclosure relates, in some aspects, to the field of biosynthetic engineering of microbes that can detect bleeding events in vivo.

### BACKGROUND OF INVENTION

Gastrointestinal (GI) bleeding, also known as gastrointestinal hemorrhaging, encompasses all forms of blood loss from the gastrointestinal tract, from the mouth to the rectum. GI bleeding is typically divided into two main types: upper gastrointestinal bleeding and lower gastrointestinal bleeding, which are not easily distinguished using current diagnostic methods. Common causes of GI bleeding include, inter alia, peptic ulcer disease, esophageal varices due to liver cirrhosis, inflammatory bowel disease, hemorrhoids, infections, cancers, vascular disorders, adverse effects of medications, and blood clotting disorders.

Typically, the diagnosis of GI bleeding is based on direct observation of blood in the stool or vomit, which can be confirmed ex vivo with a fecal occult blood test, such as a guaiac test. The guaiac test is qualitative, as guaiac, a phenol compound present in wood, turns blue in the presence of hemoglobin and hydrogen peroxide. Furthermore, dietary peroxidases (e.g., vitamin C) can give a false positive results. This and other methods for detecting GI bleeding, including endoscopy, fecal immunochemical tests, and gastric aspiration have significant disadvantages. For example, they either do not operate in vivo, are not sensitive for upper gastrointestinal bleeds, are not specific, or are inconvenient. Thus, there is a need for additional strategies, for detecting GI bleeding.

### SUMMARY OF INVENTION

This disclosure provides, inter alia, non-naturally occurring engineered microorganisms, including bacteria, that specifically detect biomedically relevant molecules to monitor gastrointestinal bleeding and to record bleeding events in cellular memory. This disclosure also provides methods for detecting bleeding events and/or treating bleeding in vivo.

Microorganisms such as bacteria (e.g., non-pathogenic) functionalized with synthetic gene circuits present a promising means to monitor human health. Previous work in synthetic biology has focused on the creation of novel genetic circuits that can integrate both logic and memory in response to environmental stimuli. Described herein are autonomous whole-cell biosensors that can be used to monitor gastrointestinal bleeding and record bleeding events in cellular memory. Such biosensors can be used to detect GI bleeding events in vivo and report bleeding events by expressing reporter molecules and/or recording bleeding events in cellular memory. Further, the engineered microorganisms, provided herein, can be used to express therapeutic molecules in response to bleeding events

The present disclosure includes the unexpected finding that E. coli engineered to express the heme-responsive transcription factor HrtR from Lactococcus lactis, the outer-membrane heme transporter ChuA, and a chimeric gene circuit responsive to HrtR could detect heme in the sub-micromolar range in vitro. The present disclosure further includes the unexpected finding that the engineered E. coli could detect gastrointestinal bleeding in vivo using two different mouse models of gastrointestinal bleeding.

Thus, the present disclosure, in some aspects, includes an engineered microorganism comprising a heme-responsive transcription factor and a genetic circuit responsive to the heme-responsive transcription factor. In some embodiments, the engineered microorganism further comprises a heme transporter. In another embodiment, the engineered microorganism's heme-responsive transcription factor is a TetR-family transcriptional repressor. In some embodiments, the engineered microorganism's heme-responsive transcription factor is from Lactococcus lactis. In other embodiments, the engineered microorganism's heme-responsive transcription factor is HrtR.

In some embodiments, the engineered microorganism is Escherichia coli or Lactococcus lactis. In other embodiments, the microorganism is Escherichia coli MG1655 or Nissle 1917.

In another embodiment, the engineered microorganism's heme transporter is from a gram negative bacterium. In some embodiments, the engineered microorganism's heme transporter is ChuA.

In some embodiments, the engineered microorganism's genetic circuit comprises a first promoter that is operably linked to a nucleic acid sequence encoding an output molecule, and wherein the first promoter is responsive to the heme-responsive transcription factor. In other embodiments, the engineered microorganism's first promoter is a P_{HrtAB(HrtR)} promoter or a P_{L} promoter with one or more HrtO operator sites. In another embodiment, the engineered microorganism further comprises a second promoter that is operably linked to a nucleic acid encoding the heme-responsive transcription factor. In some embodiments, the nucleic acid encoding the heme-responsive transcription factor encodes a ribosomal binding site (RBS) comprising the nucleic acid sequence SEQ ID NO: 17, SEQ ID NO: 18, SEQ ID NO: 19, or SEQ ID NO: 20. In some embodiments, the engineered microorganism further comprises a third promoter that is operably linked to a nucleic acid encoding the heme transporter. In some embodiments, the engineered microorganism's second and/or third promoter is constitutive. In other embodiments, the engineered microorganism's second and/or third promoter is inducible.

In some embodiments, the engineered microorganism's heme-responsive transcription factor is a response regulator from a two-component system. In another embodiment, the engineered microorganism's heme-responsive transcription factor is HssR. In other embodiments, the engineered microorganism further comprises a heme-responsive histidine kinase from a two-component system. In some embodiments, the engineered microorganism's heme-responsive histidine kinase from a two-component system is HssS. In some embodiments, the engineered microorganism's genetic circuit comprises a first promoter that is operably linked to a nucleic acid sequence encoding an output molecule, wherein the first promoter is responsive to the response regulator from a two-component system.

In some embodiments, the engineered microorganism's first promoter is a P_{HrtAB(HssR)} promoter. In other embodiments, the engineered microorganism further comprises a second promoter that is operably linked to a nucleic acid encoding the response regulator from a two-component system. In another embodiment, the engineered microorganism further comprises a third promoter that is operably linked to a nucleic acid encoding the heme-responsive histidine kinase from a two-component system. In some embodiments, the engineered microorganism's second and/or third promoter is constitutive. In another embodiment, the engineered microorganism's second and/or third promoter is inducible. In some embodiments, the engineered microorganism's output molecule is a nucleic acid, a reporter polypeptide, a recombinase, or a therapeutic protein. In another embodiment, the engineered microorganism further comprises a fourth promoter operably linked to a second output molecule, wherein the fourth promoter is responsive to the recombinase.

In some embodiments, the engineered microorganism comprises a first plasmid that encodes the heme-responsive transcription factor and the genetic circuit responsive to the heme-responsive transcription factor. In some embodiments, the first plasmid further encodes the heme transporter.

In some embodiments, the engineered microorganism comprises a first plasmid that encodes the heme-responsive transcription factor, and a second plasmid that encodes the genetic circuit responsive to the heme-responsive transcription factor. In some embodiments, the first plasmid further encodes the heme transporter. In some embodiments, the engineered microorganism comprises a third plasmid encoding the heme transporter.

In some aspects, the present disclosure further includes a method of detecting and/or treating bleeding in a subject comprising administering to the subject the engineered microorganism described above. In some embodiments, the subject has, or is at risk of having a gastrointestinal bleed. In some embodiments, the subject is administered an agent that causes gastrointestinal bleeding. For example, the agent that causes gastrointestinal bleeding may be dextran sulfate sodium (DSS) or indomethacin. In other embodiments, the subject has, or is at risk of having a disease or disorder. In another embodiment, the disease or disorder is colitis, peptic ulcer disease, liver cirrhosis, inflammatory bowel disease, hemorrhoids, an infection, cancer, a vascular disorder, an adverse effect of a medication, or a blood clotting disorder.

In some embodiments, the engineered microorganism is administered orally. In another embodiment, the engineered microorganism is administered in the form of a pill.

In other embodiments, the method further comprises obtaining the engineered microorganism from the subject and analyzing the engineered microorganism in vitro. In some embodiments, the engineered microorganism is obtained from the stool of the subject. In another embodiment, analyzing the engineered microorganism comprises polymerase chain reaction (PCR), nucleic acid sequencing, measuring the level of an output molecule, measuring fluorescence or luminescence from the engineered microorganism and/or measuring a level of the engineered microorganism from the subject. In other embodiments, the method further comprises analyzing the engineered microorganism in vivo. In some embodiments, analyzing the engineered microorganism comprises measuring fluorescence or luminescence from the engineered microorganism. In some embodiments, the analysis comprises measuring luminescence from the subject that has been administered the engineered microorganism. In another embodiment, a result of the analysis is transmitted wirelessly.

### BRIEF DESCRIPTION OF THE DRAWINGS

The accompanying drawings are not intended to be drawn to scale. For purposes of clarity, not every component may be labeled in every drawing.

FIG. 1 is a schematic of the genetically engineered (thus, non-naturally occurring) heme-responsive E. coli biosensor. Extracellular hemin is imported into the periplasm via the ChuA transporter and is imported into the cytoplasm by an unknown mechanism. Cytoplasmic heme can bind to the HrtR transcriptional repressor, which then dissociates from its cognate reporters and allows production of bioluminescence.

FIGS. 2A-2D show the hemin response of E. coli engineered to sense hemin in the presence and absence of the heme transporter ChuA. FIG. 2A shows the hemin response of an overnight culture of E. coli MG1655 transformed with HrtR+P_{hrtAB}-luxCDABE, or HrtR+ChuA+P_{hrtAB}-luxCDABE. FIG. 2B shows the hemin response of an overnight culture of E. coli MG1655 transformed with HrtR+P_{L(HrtO)}-luxCDABE or HrtR+ChuA+P_{L(HrtO)}-luxCDABE. Overnight cultures depicted in FIGS. 2A and 2B were diluted 1:100 into media induced with various concentrations of hemin. Luminescence and OD₆₀₀ measurements were taken 4 hours post-induction. FIG. 2C shows the hemin response of an overnight culture of E. coli MG1655 transformed with HrtR+ChuA+P_{hrtAB}-luxCDABE. FIG. 2D shows the hemin response of an overnight culture of E. coli MG1655 transformed with HrtR+ChuA+P_{L(HrtO)}-luxCDABE. Overnight cultures depicted in FIGS. 2C and 2D were diluted 1:100 into LB and induced with various concentrations of hemin 3.5 hours post-inoculation. Luminescence and OD₆₀₀ of the cultures were monitored every 15 minutes post-induction for 2 hours.

FIG. 3 shows the transfer curve of an improved heme-sensing circuit. The initial prototype (V1.0), using RBS3 and refined (V2.0), using RBS2 gene circuits were exposed to various concentrations of horse blood.

FIG. 4 is a schematic depicting one example of the integration of heme sensors and recombinase-based memory modules, ultimately resulting in the storage of heme-sensing in bacterial DNA.

FIG. 5 is a schematic illustrating the conversion of DNA in living cells to a digital storage media. In the top panel, invertase (recombinase) associated with the center segment of the DNA is present, resulting in inversion of the sequence between the cognate recombinase recognition sequences ([and]), resulting a “1” signal (lower panel), while the other two segments are unaffected.

FIG. 6 is a schematic depicting the analog-to-digital memory for probiotic sensors.

FIG. 7 is a schematic illustrating how heme sensors can be used to drive light expression, which can then be detected with integrated CMOS sensors equipped with ultra-low-power wireless.

FIG. 8 shows exemplary nucleic acid sequences of a natural L. lactis P_{HrtAB} promoter (SEQ ID NO: 23), and synthetic P_{L}-based promoters, P_{L(TetO)} (SEQ ID NO: 24) and P_{L(HrtO)} (SEQ ID NO: 16). The −35 in the L. lactis P_{HrtAB} sequence indicates a putative −35 site.

FIGS. 9A-9B show the hemin and blood response of E. coli engineered to sense hemin in the presence and absence of the heme transporter ChuA. FIG. 9A shows the hemin response of a culture of E. coli MG1655 RBS3 (SEQ ID NO: 19), P_{L(HrtO)}-luxCDABE only (lux); P_{L(HrtO)}-luxCDABE and HrtR together (lux+hrtR); or HrtR and ChuA and P_{L(HrtO)}-luxCDABE together (lux+hrtR+chuA). FIG. 9B shows the blood response of a culture of E. coli MG1655 transformed with P_{L(HrtO)}-luxCDABE only (lux); P_{L(HrtO)}-luxCDABE and HrtR together (lux+hrtR); or HrtR and ChuA and P_{L(HrtO)}-luxCDABE together (lux+hrtR+chuA). Overnight cultures depicted in FIGS. 9A and 9B were diluted 1:100 into media, grown for 2 hours and then induced with various concentrations of hemin (FIG. 9A) or blood (FIG. 9B).

FIGS. 10A-10B show the hemin (FIG. 10A) and blood (FIG. 10B) response of E. coli engineered with various ribosomal binding sites (RBS1-4) upstream of HrtR. Overnight cultures of E. coli MG1655 were transformed with P_{L(HrtO)}-luxCDABE-hrtR-chuA having an HrtR ribosomal binding sequence (RBS) of RBS1 (SEQ ID NO: 17), RBS2 (SEQ ID NO: 18), RBS3 (SEQ ID NO: 19), or RBS4 (SEQ ID NO: 20).

FIG. 11 shows the kinetic response of E. coli MG1655 transformed with P_{L(HrtO)} luxCDABE-hrtR_{(RBS2)}-chuA to hemin (10 μM), blood (0.1%), or an uninduced control.

FIG. 12 shows the dose response of an engineered probiotic E. coli Nissle 1917 transformed with P_{L(HrtO)}-luxCDABE-hrtR_{(RBS2)}-chuA to varying concentrations of blood.

FIGS. 13A-13B show the response of E. coli MG1655 engineered to sense hemin fed to control mice or mice treated with the nonsteroidal anti-inflammatory drug (NSAID) indomethacin. FIG. 13A shows the response of E. coli transformed with P_{L(HrtO)}-luxCDABE-hrtR_{(RBS2})-chuA to sense hemin after being fed to mice treated with 10 mg/kg indomethacin, 5 mg/kg indomethacin, or a control (0 mg/kg indomethacin). Mice were gavaged with engineered bacteria 18 hours post-indomethacin treatment, and stool was collected 6 h post-gavage to assess luminescence activity and colony counts. FIG. 13B shows the response of E. coli engineered to sense hemin that was collected from the stool of mice testing positive (guaiac +) or negative (guaiac −) for fecal occult blood in the stool.

FIG. 14 shows the response of E. coli engineered to sense hemin measured in vivo. E. coli transformed with P_{L(HrtO)}-luxCDABE-hrtR_{(RBS2)}-chuA to sense hemin was fed to control mice (0 mg/kg indomethacin) or mice treated with 5 mg/kg or 10 mg/kg indomethacin. The luminescence activity from living mice was measured using in vivo luminescence imaging techniques (top 3 images) and quantified (bottom graph).

FIG. 15 shows the response of E. coli engineered to sense hemin fed to control mice or a DSS-induced mouse colitis model of rectal bleeding. Control mice, or mice treated with 3% dextran sulfate sodium (DSS) were gavaged daily with E. coli transformed with P_{L(HrtO)}-luxCDABE-hrtR_{(RBS2)}-chuA. Fecal occult blood (FOB) was detected on day 3 following DSS treatment. The “*” indicates p<0.05, and “LOD” indicates the level of detection.

### DETAILED DESCRIPTION OF DISCLOSURE

Provided herein are engineered and thus non-naturally occurring microorganisms, and methods for detecting bleeding events and treating bleeding using such microorganisms.

### Engineered Microorganisms

Some aspects of the present disclosure are directed to engineered microorganisms having a heme-responsive transcription factor and a genetic circuit responsive to the heme-responsive transcription factor. An “engineered microorganism,” as used herein, refers to a microorganism that does not occur in nature. Engineered microorganisms of the present disclosure, in some embodiments, contain one or more exogenous nucleic acids (i.e., nucleic acids that the microorganism would not normally contain) or nucleic acids that do not occur in nature (e.g., an engineered nucleic acid encoding a heme-responsive transcription factor). Accordingly, an engineered microorganism can be a microorganism that has been designed, produced, prepared, synthesized, manufactured and/or manipulated by a human.

In some embodiments, an engineered microorganism contains an engineered nucleic acid. A “nucleic acid” is at least two nucleotides covalently linked together, which in some instances may contain phosphodiester bonds (e.g., a phosphodiester “backbone”). An “engineered nucleic acid,” as used herein, is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a murine nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified in vitro or chemically synthesized (e.g., using a nucleic acid automated synthesizer). A synthetic nucleic acid includes nucleic acids that are chemically modified, or otherwise modified, but can base pair with naturally-occurring nucleic acid molecules. Recombinant and synthetic nucleic acids also include nucleic acids that result from the replication of either of the foregoing.

In some embodiments, an engineered microorganism contains an exogenous independently-replicating nucleic acid (e.g., an engineered nucleic acid present on an episomal vector). In some embodiments, an engineered microorganism is produced by introducing a foreign or exogenous nucleic acid into a cell using methods well known in the art. A nucleic acid may be introduced into a cell by conventional methods, such as, for example, electroporation (see, e.g., Heiser W. C. Transcription Factor Protocols: Methods in Molecular BiologyTM 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, the engineered microorganisms of the present disclosure are prokaryotes (e.g., bacterial cells). In some embodiments, the engineered microorganisms are bacterial cells. Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Lactobacillus spp., Lactococcus spp., Bacillus spp., Enterobacter spp., Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Erysipelothrix spp., Salmonella spp., Staphylococcus spp., Streptomyces spp., Bacteroides spp., Prevotella spp., Clostridium spp., or Bifidobacterium spp.

In some embodiments, the engineered microorganisms are non-pathogenic bacteria that are derived from a normal internal ecosystem such as bacterial flora. In some embodiments, the engineered microorganisms are non-pathogenic bacteria that are derived from a normal internal ecosystem of the gastrointestinal tract. Non-limiting examples of non-pathogenic bacteria that are part of the normal flora in the gastrointestinal tract include bacteria from the genera Bacteroides, Clostridium, Fusobacterium, Eubacterium, Ruminococcus, Peptococcus, Peptostreptococcus, Bifidobacterium, Escherichia and Lactobacillus.

In some embodiments, bacterial cells of the disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, the engineered microorganisms are lactic acid bacteria (LAB). “Lactic acid bacteria,” as used herein, refer to Gram-positive, non-spore forming cocci, coccobacilli or rods with low GC content (i.e., a DNA base composition of less than 53 mol % G+C). Lactic acid bacteria generally are non-respiratory and lack catalase. Typically, lactic acid bacteria ferment glucose primarily to lactic acid, or to lactic acid, CO₂ and ethanol. In some embodiments, the lactic acid bacteria are, without limitation, Lactococcus lactis, Lactobacillus acidophilus, Lactobacillus gasseri, Leuconostoc lactis, Lactobacillus brevis, Lactobacillus plantarum, Lactobacillus casei, Lactobacillus gasseri, Lactobacillus helveticus, Streptococcus pyogenes, Streptococcus agalactiae, Streptococcus pneumoniae, or Streptococcus zooepidemicus.

In some embodiments, the engineered microorganisms are Lactococcus lactis. In some embodiments the engineered microorganisms are Escherichia coli (E. coli). In some embodiments, the engineered microorganisms are E. coli strain MG1655.

In some embodiments, the engineered microorganisms, provided herein comprise a heme-responsive transcription factor. A “heme-responsive transcription factor” as used herein refers to a molecule that causes a change in transcriptional activity in response to heme, either by binding heme directly or via signal transduction following the activation of another molecule (e.g., a heme binding receptor of a two-component system) that binds heme. As used herein, the term “heme” refers to an iron (e.g., Fe²⁺ or Fe³⁺) containing porphyrin (e.g., tetrapyrrole), or any analog thereof (e.g., hemin).

The heme-responsive transcription factors of the present disclosure, in some embodiments, are comprised of protein. The terms “protein,” “peptide,” and “polypeptide” are used interchangeably herein and refer to a polymer of amino acid residues linked together by peptide (amide) bonds. A protein, peptide, or polypeptide may refer to an individual protein or a collection of proteins.

The heme-responsive transcription factors of the present disclosure, in some embodiments, are transcriptional activators or transcriptional repressors that are either activated or repressed in response to binding heme. In some embodiments, the heme-responsive transcription factor is a transcriptional activator. In some embodiments, the heme-responsive transcriptional activator is active when bound to heme. In some embodiments, the heme-responsive transcriptional activator is inactive or has decreased activity when bound to heme. In some embodiments, the heme-responsive transcription factor is a transcriptional repressor. In some embodiments, the heme-responsive transcriptional repressor is active when bound to heme. In some embodiments, the heme-responsive transcriptional repressor is inactive or has decreased activity when bound to heme.

The heme-responsive transcription factors of the present disclosure, in some embodiments, are transcriptional repressors that are inhibited upon binding of heme. For example, when the heme-responsive transcriptional repressor is not bound to heme, it represses transcriptional activity (e.g., of a promoter, which may be operably linked to a nucleic acid sequence). Conversely, when the heme-responsive transcriptional repressor binds heme, the repressor activity is decreased, thereby permitting transcription. In some embodiments, the heme-responsive transcription factor is a TetR-family transcriptional repressor. The “TetR family of transcriptional repressors” refers to a family of transcriptional repressors that can be identified by amino acid sequence homology to members of the TetR family, including TetR, QacR, CprB and EthR. The stretch that best defines the profile of this family is made up of 47 amino acid residues that correspond to the helix-turn-helix DNA binding motif and adjacent regions in the three-dimensional structures of TetR, QacR, CprB, and EthR, four family members for which the function and three-dimensional structure are known. The TetR family is named after the member of this group that has been most completely characterized genetically and biochemically, the TetR protein. Typically, members of the TetR family of repressors are identified by amino acid sequence profile which can be easily used to recognize TetR family members in SWISS-PROT and TrEMBL and in all available proteins from prokaryotic genome sequences as described in Juan L. Ramos, J. L., et al., “The TetR Family of Transcriptional Repressors” Microbiol Mol Biol Rev. 2005 June; 69(2): 326-356; the contents of which are hereby incorporated by reference. Accordingly, proteins belonging to the TetR family of transcriptional repressors would be apparent to one of skill in the art. Exemplary members of the TetR family of transcriptional repressors include, without limitation, HrtR.

In some embodiments, the heme-responsive transcription factor comprises HrtR. HrtR refers to a naturally-occurring intracellular heme binding protein that regulates transcription in response to heme. HrtR is conserved among numerous commensal bacteria. Thus, in some embodiments, the heme-responsive transcription factor is an HrtR from any naturally-occurring microorganism (e.g., any of the microorganisms provided herein). HrtR proteins from naturally-occurring microorganisms are known in the art and would be apparent to the skilled artisan. For example, HrtR from Lactococcus lactis has been described by Lechardeur D., et al., “Discovery of Intracellular Heme-binding Protein HrtR, Which Controls Heme Efflux by the Conserved HrtB-HrtA Transporter in Lactococcus lactis” J Biol Chem. 2012 Feb. 10; 287(7): 4752-4758; the contents of which are hereby incorporated by reference for its description of HrtR. In some embodiments, the heme-responsive transcription factor comprises a protein that is at least 70%, at least 75%, at least 80%, at least 85%, at least 90%, at least 95%, at least 98%, or at least 99% identical to a naturally-occurring HrtR protein. In some embodiments, the heme-responsive transcription factor comprises an HrtR protein from Lactococcus lactis. In some embodiments, the heme-responsive transcription factor comprises the amino acid sequence of SEQ ID NO: 1. In some embodiments, the heme-responsive transcription factor consists essentially of the amino acid sequence of SEQ ID NO: 1. In some embodiments, the heme-responsive transcription factor consists of the amino acid sequence of SEQ ID NO: 1.

In some embodiments, the engineered microorganism of the present disclosure comprises a heme transporter. A “heme transporter” as used herein, refers to a molecule, typically comprised of protein, that allows the transport of heme across a cell membrane. Without wishing to be bound by any particular theory, extracellular heme may not readily cross the cell membrane in some microorganisms (e.g., gram negative microorganisms) making the intracellular heme-responsive transcription factor less sensitive to heme. Thus, in some embodiments, a heme transporter is expressed in the engineered microorganism to allow extracellular heme to enter the engineered microorganism more readily, where it can bind a heme-responsive transcription factor and modulate transcription in the engineered microorganism. Heme transporters are known in the art and would be recognized by the skilled artisan. For example, exemplary heme transporters have been described previously in Tong Y., “Bacterial heme-transport proteins and their heme-coordination modes” Arch Biochem Biophys. 2009 Jan. 1; 481(1): 1-15; the contents of which are hereby incorporated by reference. Exemplary heme transporters include, without limitation, ChuA. However, it should be appreciated that the heme transporters described herein and in the cited references are exemplary and are not meant to be limiting. In some embodiments, the heme transporter is from a gram-negative bacteria. Typically, gram-negative bacteria are a group of bacteria that do not retain the crystal violet stain used in the Gram staining due to a difference in cell wall structure between gram-positive and gram-negative bacteria. In some embodiments, the heme transporter comprises a protein that is at least 70%, at least 75%, at least 80%, at least 85%, at least 90%, at least 95%, at least 98%, or at least 99% identical to a naturally-occurring heme transporter. In some embodiments, the heme transporter comprises a ChuA protein from E. coli. In some embodiments, the heme transporter comprises the amino acid sequence of SEQ ID NO: 2. In some embodiments, the heme transporter consists essentially of the amino acid sequence of SEQ ID NO: 2. In some embodiments, heme transporter consists of the amino acid sequence of SEQ ID NO: 2.

In some embodiments, the heme-responsive transcription factor is a response regulator from a two-component system. In some embodiments, the engineered microorganisms of the present disclosure further comprise a heme-responsive histidine kinase from a two-component system. Two-component regulatory systems serve as a basic stimulus-response coupling mechanism to allow organisms to sense and respond to changes in many different environmental conditions. See e.g., Stock A. M., et al., “Two-component signal transduction,” Annu. Rev. Biochem., 2000, 69 (1): 183-215, the contents of which are hereby incorporated by reference. Typically two-component systems include a membrane-bound histidine kinase (e.g., HssS) that senses a specific environmental stimulus (e.g., heme) and a corresponding response regulator (e.g., HssR) that mediates the cellular response (e.g., through differential expression of target genes). In some embodiments, the heme-responsive transcription factor is from an HssRS two-component system. In some embodiments, the heme-responsive histidine kinase is from an HssRS two-component system. Heme sensing two-component systems (e.g., HssRS), which include response regulators and histidine kinases are known in the art and have been described previously in Stauff D. L., et al., “The heme sensor system of Staphylococcus aureus” Contrib Microbiol. 2009; 16:120-35; and in Stauff D. L., et al., “Signaling and DNA-binding activities of the Staphylococcus aureus HssR-HssS two-component system required for heme sensing” J Biol Chem. 2007 Sep. 7; 282(36):26111-21; the contents of each of which are hereby incorporated by reference for the description of heme sensing two-component systems. However, it should be appreciated that the heme-responsive transcription factors and the heme-responsive histidine kinases may be from any two-component system and the exemplary two-component systems described herein and in the cited references are not meant to be limiting.

In some embodiments, the heme-responsive transcription factor comprises HssR. HssR refers to a naturally-occurring response regulator from an HssRS two-component system that regulates transcription in response to heme. In some embodiments, the heme-responsive transcription factor is an HssR protein from any naturally-occurring microorganism (e.g., any of the microorganisms provided herein). HssR proteins from naturally-occurring microorganisms are known in the art and would be apparent to the skilled artisan. In some embodiments, the heme-responsive transcription factor comprises a protein that is at least 70%, at least 75%, at least 80%, at least 85%, at least 90%, at least 95%, at least 98%, or at least 99% identical to a naturally-occurring HssR protein. In some embodiments, the heme-responsive transcription factor comprises an HssR protein from Staphylococcus aureus. In some embodiments, the heme-responsive transcription factor comprises the amino acid sequence of SEQ ID NO: 3. In some embodiments, the heme-responsive transcription factor consists essentially of the amino acid sequence of SEQ ID NO: 3. In some embodiments, the heme-responsive transcription factor consists of the amino acid sequence of SEQ ID NO: 3.

In some embodiments, the heme-responsive histidine kinase comprises HssS. HssS refers to a naturally-occurring histidine kinase from an HssRS two-component system that regulates transcription in response to heme. In some embodiments, the heme-responsive histidine kinase is an HssS protein from any naturally-occurring microorganism (e.g., any of the microorganisms provided herein). HssS proteins from naturally-occurring microorganisms are known in the art and would be apparent to the skilled artisan. In some embodiments, the heme-responsive transcription factor comprises a protein that is at least 70%, at least 75%, at least 80%, at least 85%, at least 90%, at least 95%, at least 98%, or at least 99% identical to a naturally-occurring HssS protein. In some embodiments, the heme-responsive transcription factor comprises an HssS protein from Lactococcus lactis. In some embodiments, the heme-responsive transcription factor comprises the amino acid sequence of SEQ ID NO: 4. In some embodiments, the heme-responsive transcription factor consists essentially of the amino acid sequence of SEQ ID NO: 4. In some embodiments, the heme-responsive transcription factor consists of the amino acid sequence of SEQ ID NO: 4.

The disclosure further provides variants of any of the heme-responsive transcription factor amino acid sequences, any of the heme-responsive histidine kinase amino acid sequences or any of the heme transporter amino acid sequences described herein. As used herein, a variant of a heme-responsive transcription factor amino acid sequence, a heme-responsive histidine kinase amino acid sequence or a heme transporter amino acid sequence is an amino acid sequence that is not identical to, but shares a degree of homology with the heme-responsive transcription factor amino acid sequences, the heme-responsive histidine kinase amino acid sequences, or the heme transporter amino acid sequences respectfully described herein As used herein, the term “homology” refers to the overall relatedness between proteins. In some embodiments, proteins are considered to be “homologous” to one another if their amino acid sequences are at least 70%, at least 75%, at least 80%, at least 85%, at least 90%, at least 95%, or at least 99% identical as determined by standard methods of comparing sequences used in the art, such as the BLAST (Basic Local Alignment Search Tool) programs of the National Center for Biotechnology Information, using default parameters. Accordingly, proteins that are homologous to any of the heme-responsive transcription factor amino acid sequences (e.g., amino acid sequences of HrtR and HssR), heme-responsive histidine kinase amino acid sequences (e.g., amino acid sequences of HssS), or heme transporter amino acid sequences (e.g., amino acid sequences of ChuA), described herein, are also within the scope of this disclosure.

### Genetic Circuits

In some embodiments, the engineered microorganisms of the present disclosure comprise genetic circuits responsive to any of the heme-responsive transcription factors, provided herein. A “genetic circuit,” as used herein, refers to a functional cluster of genes or nucleic acids that impact each other's expression through inducible transcription factors or cis-regulatory elements. A genetic circuit is “responsive to a heme-responsive transcription factor” if the heme-responsive transcription factor modulates the expression of at least one nucleic acid or gene of the genetic circuit. Activation or repression of transcription of a nucleic acid or gene can occur via direct binding of heme to a heme-responsive transcription factor (e.g., HrtR). Alternatively, activation or repression of transcription of a nucleic acid or gene can occur via signal transduction following activation of a heme-responsive histidine kinase in response to binding a ligand (e.g., heme). For example, phosphorylation of a heme-responsive histidine kinase (e.g., in response to binding a ligand such as heme) may phosphorylate a heme-responsive transcription factor to activate or repress transcription of a nucleic acid or gene of the genetic circuit. Without wishing to be bound by any particular theory, signal transduction may occur through the transfer of phosphoryl groups from adenosine triphosphate (ATP) to a specific histidine residue in the heterologous histidine kinases (e.g., by an autophosphorylation reaction). Molecules referred to as response regulators (e.g., HssR) may then be phosphorylated on an aspartate residue. Phosphorylation of the response regulators can cause a change in the conformation of the response regulators, typically activating an attached output domain, which then may lead to the activation or repression of expression of target genes or nucleic acids. Accordingly, in some embodiments, a gene circuit comprises a gene that is transcriptionally activated when a heme-responsive transcription factor is bound by a ligand (e.g., heme), or when a heme-responsive transcription factor is activated (e.g., by phosphorylation) via signal transduction by a heme-responsive histidine kinase. In some embodiments, a gene circuit comprises a gene that is transcriptionally repressed when a heme-responsive transcription factor is bound by a ligand (e.g., heme), or when a heme-responsive transcription factor is activated (e.g., by phosphorylation) via signal transduction by a heme-responsive histidine kinase.

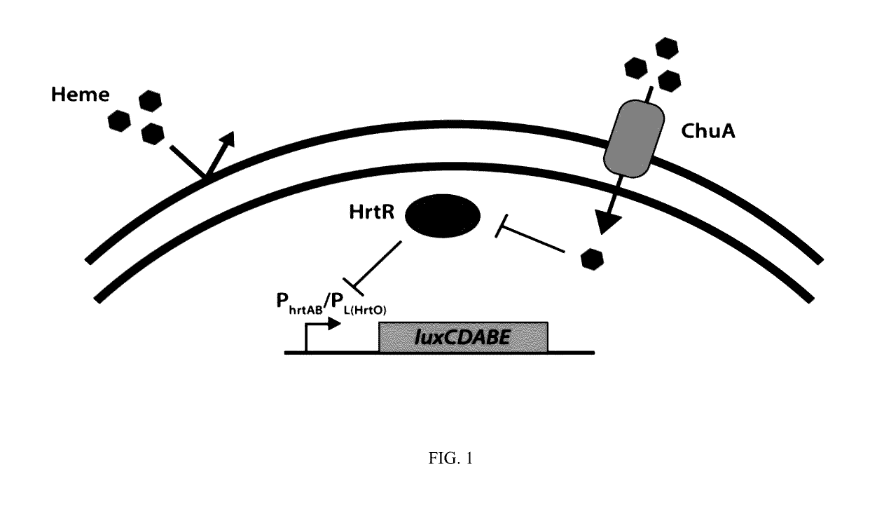

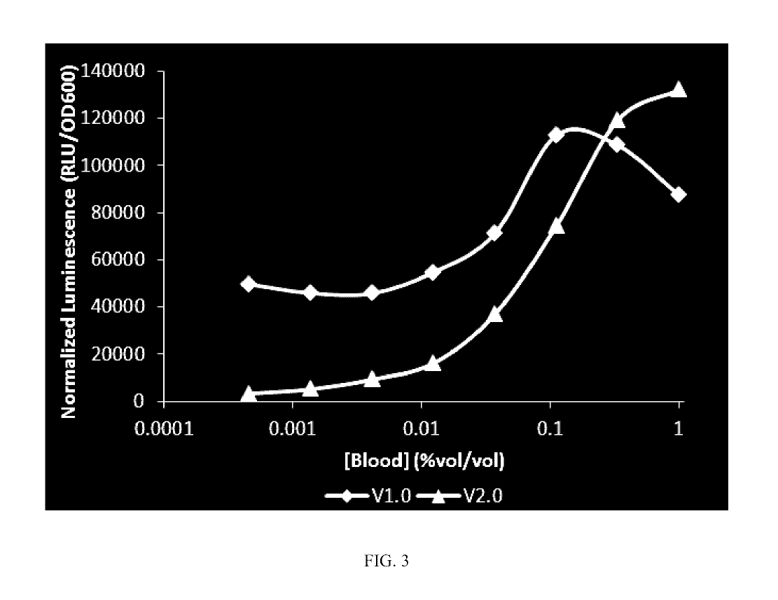

In some embodiments the genetic circuit comprises a first promoter that is operably linked to a nucleic acid sequence encoding an output molecule, wherein the first promoter is responsive to the heme-responsive transcription factor. As one non-limiting example, in response to binding heme, the transcriptional repressor HrtR is inhibited, thus allowing transcription of a luxCDABE output molecule (see e.g., FIG. 1). As another non-limiting example, in response to binding heme, the heme-responsive histidine kinase HssS phosphorylates and activates the heme-responsive transcription factor HssR, thus promoting transcription of a b×b output molecule (see e.g., FIG. 4). It should be appreciated that the genetic circuits, described herein, may comprise one or more nucleic acids which may or may not be linked.

The genetic circuits of the present disclosure may comprise one or more promoters operably linked to a nucleotide sequence encoding, for example, an output molecule. A “promoter” refers to a control region of a nucleic acid sequence at which initiation and rate of transcription of the remainder of a nucleic acid sequence are controlled. A promoter may also contain sub-regions to which regulatory proteins and molecules may bind, such as RNA polymerase and other transcription factors. Promoters may be constitutive, inducible, activatable, repressible, or any combination thereof. In some embodiments, the genetic circuit comprises at least 1 at least 2, at least 3, at least 4, at least 5, at least 6, at least 7, at least 8, at least 9, at least 10, at least 15, at least 20, at least 30 or at least 50 promoters. In some embodiments one or more of the promoters may be a P_{HrtAB(HrtR)} promoter, a P_{HrtAB(HssR)} promoter and/or a P_{L} promoter. As used herein, a P_{HrtAB(HrtR)} promoter is a promoter that is responsive to HrtR. As used herein, a P_{HrtAB(HssR)} promoter is a promoter that is responsive to HssR. In some embodiments one or more of the promoters comprises SEQ ID NOs: 12, 13, and/or 14. In some embodiments one or more of the promoters consists of SEQ ID NOs: 12, 13, and/or 14. In some embodiments one or more of the promoters consists essentially of SEQ ID NOs: 12, 13, and/or 14.

In some embodiments the genetic circuits of the present disclosure comprise one or more operator sites. An “operator site” as used herein, refers to a segment of DNA to which a molecule (e.g., a transcriptional repressor) binds to regulate transcription or gene expression. An operator site, in some embodiments, is associated with one or more promoters to modulate transcription from the one or more promoters. In some embodiments the genetic circuits, described herein, comprise one or more operator sites. In some embodiments, the genetic circuit comprises at least 1 at least 2, at least 3, at least 4, at least 5, at least 6, at least 7, at least 8, at least 9, at least 10, at least 15, at least 20, at least 30 or at least 50 operator sites. In some embodiments one or more of the operator sites may be an HrtO operator site. In some embodiments one or more of the operator sites comprise the nucleic acid sequence of SEQ ID NO: 15. In some embodiments one or more of the operator sites consist of the nucleic acid sequence of SEQ ID NO: 15. In some embodiments one or more of the operator sites consist essentially of the nucleic acid sequence of SEQ ID NO: 15. In some embodiments, a promoter is associated with an operator site. In some embodiments, a promotor with an operator site comprises the nucleic acid sequence of SEQ ID NO: 16. In some embodiments, a promotor with an operator site consists of the nucleic acid sequence of SEQ ID NO: 16. In some embodiments, a promotor with an operator site consists essentially of the nucleic acid sequence of SEQ ID NO: 16.

A promoter drives expression or transcription of the nucleic acid sequence to which it is operatively linked. In some embodiments, the promoter is operably linked to a nucleic acid encoding a hybrid receptor or an output molecule. A promoter is considered to be “operably linked” when it is in a correct functional location and orientation in relation to the nucleic acid sequence it regulates, thereby resulting in the ability of the promoter to drive transcription initiation or expression of that sequence.

A promoter may be one naturally associated with a gene or sequence, as may be obtained by isolating the 5′ non-coding sequences located upstream of the coding segment of a given gene or sequence (e.g., an endogenous promoter).

In some embodiments, a coding nucleic acid sequence may be positioned under the control of a recombinant or heterologous promoter, which refers to a promoter that is not normally associated with the coding sequence in its natural environment. Such promoters may include promoters of other genes; promoters isolated from another cell type; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. No. 4,683,202 and U.S. Pat. No. 5,928,906).

In some embodiments, the promoters described herein are “constitutive promoters,” which are promoters that are constitutively active in the cell (i.e., not regulated in response to specific stimuli). Constitutive promoters (e.g., constitutive bacterial promoters) are known in the art and include, without limitation, P32, P57, P59, Pxyl, PclpB, PrepU and PlepA.

In some embodiments, the promoters described herein are “inducible promoters,” which are promoters that are active or inactive in response to a particular stimulus, condition, or an inducer signal. An inducer signal may be endogenous or a normally exogenous condition (e.g., light), compound (e.g., chemical or non-chemical compound) or protein that contacts an inducible promoter in such a way as to activate transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve direct activation of or indirect activation of a promoter as may occur by inactivation of a repressor molecule that prevents transcription from the promoter. A “repressor molecule” is any molecule that can bind to a promoter and prevent transcription of a gene or nucleic acid sequence to which the promoter is operably linked. Conversely, deactivation of transcription may involve direct action on a promoter to prevent transcription or indirect action on a promoter by activating a repressor that then acts on the promoter.

The administration or removal of an inducer signal results in a switch between activation and inactivation of the transcription of the operably linked nucleic acid sequence. Thus, the active state of a promoter operably linked to a nucleic acid sequence refers to the state in which the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Other inducible promoter systems are known in the art and may be used in accordance with the present disclosure.

In some embodiments, inducible promoters of the present disclosure function in prokaryotic cells (e.g., bacterial cells). Examples of inducible promoters for use in prokaryotic cells include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), GS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLacO1, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), σS promoters (e.g., Lutz-Bujard LacO with alternative sigma factor σ38), σ32 promoters (e.g., Lutz-Bujard LacO with alternative sigma factor σ32), and σ54 promoters (e.g., glnAp2); negatively regulated B. subtilis promoters such as repressible B. subtilis GA promoters (e.g., Gram-positive IPTG-inducible, Xyl, hyper-spank) and σB promoters. Other inducible microbial promoters may be used in accordance with the present disclosure.

In some embodiments, the engineered microorganisms, provided herein, comprise a second promoter that is operably linked to a nucleic acid encoding a heme-responsive transcription factor. In some embodiments, the heme-responsive transcription factor is any of the heme-responsive transcription factors, provided herein, that bind heme directly (e.g., HrtR). In some embodiments, the engineered microorganisms, provided herein, comprise a third promoter that is operably linked to a nucleic acid encoding a heme transporter (e.g., any of the heme transporters, described herein. In some embodiments the second and/or third promoters are inducible or constitutive.

In some embodiments, the engineered microorganisms, provided herein, comprise a second promoter that is operably linked to a nucleic acid encoding a response regulator from a two-component system. In some embodiments, the response regulator is any of the response regulators, provided herein, that is responsive to a heme-responsive histidine kinase (e.g., HssR). In some embodiments, the engineered microorganisms, provided herein, comprise a third promoter that is operably linked to a nucleic acid encoding a heme-responsive histidine kinase from a two-component system. In some embodiments, the heme-responsive histidine kinase is any of the heme-responsive histidine kinases, provided herein (e.g., HssS). In some embodiments the second and/or third promoters are inducible or constitutive.

In some embodiments, the genetic circuits, provided herein, comprise a first promoter that is operably linked to a nucleic acid sequence encoding an output molecule, wherein the first promoter is responsive to the heme-responsive transcription factor. The term “output molecule,” as used herein refers to a nucleic acid or protein that is expressed in response to the state of the heme-responsive transcription factor. In some embodiments, the output molecule is expressed when the heme-responsive transcription factor is bound to a ligand (e.g., heme), or when a heme-responsive histidine kinase that activates the heme-responsive transcription factor is bound to a ligand (e.g., heme). In some embodiments, the output molecule is expressed when the heme-responsive transcription factor is not bound to a ligand (e.g., heme), or when a heme-responsive histidine kinase that activates the heme-responsive transcription factor is not bound to a ligand (e.g., heme).

In some embodiments, the output molecule is a nucleic acid, a reporter polypeptide, a recombinase, or a therapeutic protein. In some embodiments, the output molecule is a reporter polypeptide. In some embodiments, the reporter polypeptide is a fluorescent polypeptide. Fluorescent polypeptides include, without limitation cyan fluorescent protein (e.g., AmCyan1), green fluorescent protein (e.g., EGFP, AcGFP1, and ZsGreen1), yellow fluorescent protein (e.g., ZsYellow1 and mBananna), orange fluorescent protein (e.g., mOrange and mOrange2), red fluorescent protein (e.g., DsRed, tdTomato, mStrawberry and mCherry), and far-red fluorescent protein (e.g., HcRed1, mRaspberry and mPlum). In some embodiments, the reporter polypeptide is luxCDABE. In some embodiments the reporter polypeptide comprises luxC, luxD, luxA, luxB, and/or luxE. In some embodiments, the reporter polypeptide comprises one or more of the amino acid sequences of SEQ ID NOs: 5, 6, 7, 8, or 9. In some embodiments, the reporter polypeptide is a green fluorescent protein. In some embodiments, the reporter polypeptide comprises the amino acid sequence of SEQ ID NO: 11. It should be appreciated that reporter peptides, described herein, are not meant to be limiting and that additional reporter peptides are within the scope of this disclosure.

In some embodiments, the output molecule is a nucleic acid. In some embodiments the output molecule is a ribonucleic acid (RNA). In some embodiments the RNA output molecule is part of a molecular reporting system, such as a reporting system described in Gredell J. A., “Protein and RNA engineering to customize microbial molecular reporting”, Biotechnol J. 2012 April; 7(4):477-99; the contents of which are hereby incorporated by reference. Additional nucleic acid output molecules are within the scope of this disclosure.

In some embodiments, the output molecule is a therapeutic protein. In some embodiments, the therapeutic protein is an anti-inflammatory peptide. An anti-inflammatory peptide, as used herein refers to a peptide capable of reducing inflammation. Anti-inflammatory peptides are well known in the art and would be apparent to a skilled artisan. Exemplary anti-inflammatory peptides include, without limitation, phospholipase A2s, for example PLA2, and various anti-inflammatory cytokines or inhibitors of pro-inflammatory cytokines. However, the anti-inflammatory peptides, described herein, are exemplary and not meant to be limiting. Accordingly, additional anti-inflammatory peptides are within the scope of this disclosure. In some embodiments, the therapeutic protein is a coagulant peptide. A coagulant peptide, as used here, refers to a peptide capable of coagulating blood, for example at the site of a bleed. Coagulant peptides are well known in the art and would be apparent to a skilled artisan. Exemplary coagulant peptides include, without limitation, clotting factors, for example, Factor V (FV), Factor FVII (FVII), Factor VIII (FVIII), Factor IX (FIX), Factor X (FX), Factor XI (FXI), Factor XII (FXII), Factor XIII (FXIII), and von Willebrand Factor (vWF). However, the coagulant peptides, described herein, are exemplary and not meant to be limiting. Accordingly, additional coagulant peptides are within the scope of this disclosure. In some embodiments, the output molecule is a member of a pathway that makes a small molecule drug. Such molecules are known in the art and would be apparent to the skilled artisan.

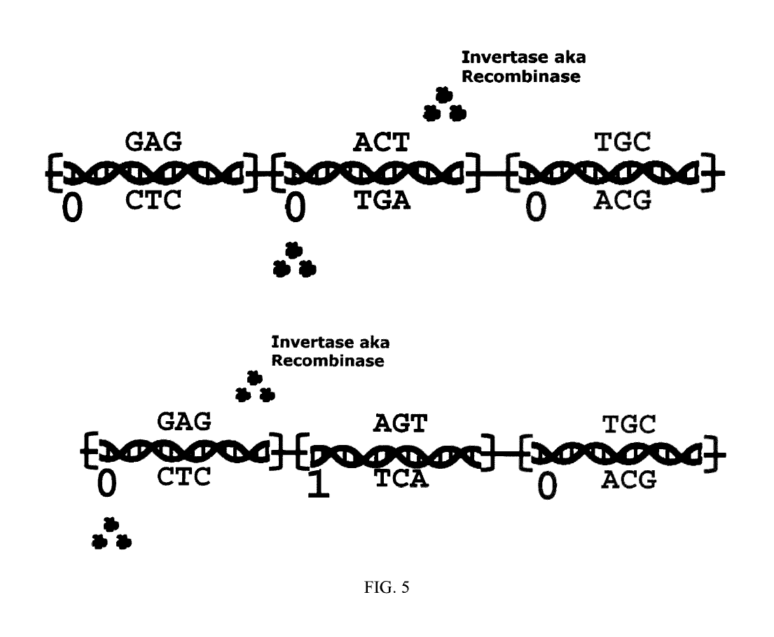

In some embodiments, the output molecule is a recombinase. In some embodiments, the recombinases are used to impart stable, DNA-base memory to logic and memory systems within the engineered microorganisms. Recombinase-based logic and memory systems are known in the art and have been described in U.S. Patent Application Publication #US-2014-0315310-A1 (published on Oct. 23, 2014), and in PCT Application Publication #WO2014/093852 (published on Jun. 19, 2014), the contents of each of which are hereby incorporated by reference for their description of recombinase-based logic and memory systems. In some embodiments the engineered microorganisms of the present disclosure comprise any of the recombinase-based logic and memory systems described in the references provided herein. In some embodiments, the logic and memory systems provided are used to record bleeding events in cellular memory or to control expression of other output proteins such as is depicted in FIG. 6.

In some embodiments the output molecule is a recombinase. A “recombinase,” as used herein, is a site-specific enzyme that recognizes short DNA sequence(s), which sequence(s) are typically between about 30 base pairs (bp) and 40 bp, and that mediates the recombination between recombinase recognition sequences, which results in the excision, integration, inversion, or exchange of DNA fragments between the recombinase recognition sequences.

Recombinases can be classified into two distinct families: serine recombinases (e.g., resolvases and invertases) and tyrosine recombinases (e.g., integrases), based on distinct biochemical properties. Serine recombinases and tyrosine recombinases are further divided into bidirectional recombinases and unidirectional recombinases. Examples of bidirectional serine recombinases include, without limitation, β-six, CinH, ParA and γδ; and examples of unidirectional serine recombinases include, without limitation, Bxb1, φC31, TP901, TG1, φBT1, R4, φRV1, φFC1, MR11, A118, U153 and gp29. Examples of bidirectional tyrosine recombinases include, without limitation, Cre, FLP, and R; and unidirectional tyrosine recombinases include, without limitation, Lambda, HK101, HK022 and pSAM2. The serine and tyrosine recombinase names stem from the conserved nucleophilic amino acid residue that the recombinase uses to attack the DNA and which becomes covalently linked to the DNA during strand exchange. In some embodiments, the output molecule is Bxb1. In some embodiments, the output molecule comprises the amino acid sequence of SEQ ID NO: 10.

Also provided herein are vectors comprising any of the engineered nucleic acids described herein. In some embodiments vectors comprise any of the heme-responsive transcription factors, any of the heme transporters, any of the heme-responsive transcription factors from a two-component system, or any of the heme-responsive histidine kinases, described herein. In some embodiments, vectors comprise any of the genes, nucleic acids, and/or promoters of any of the genetic circuits described herein. In some embodiments, vectors comprise any of the output molecules described herein. A “vector” is a nucleic acid (e.g., DNA) used as a vehicle to artificially carry genetic material (e.g., an engineered nucleic acid) into a cell where, for example, the nucleic acid can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmids typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector.

### Applications

Aspects of the disclosure relate to methods for detecting and/or treating bleeding in a subject comprising administering to the subject any of the engineered microorganisms provided herein. In some embodiments, the methods are for detecting bleeding in a subject. In some embodiments, the methods are for treating bleeding in a subject. The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is not a normal subject or healthy volunteer.

In some embodiments, the subject has or is at risk of having a gastrointestinal bleed. In some embodiments, the subject has or is at risk of having a disease or disorder. In some embodiments, the disease or disorder is peptic ulcer disease, liver cirrhosis, inflammatory bowel disease, hemorrhoids, an infection, cancer, a vascular disorder, an adverse effect of a medication, or a blood clotting disorder. In some embodiments, the subject has or is at risk of having inflammatory bowel disease Inflammatory bowel disease (IBD) refers to a group of inflammatory conditions of the small intestine and colon. In some embodiments, the IBD is Crohn's disease, ulcerative colitis, collagenous colitis, lymphocytic colitis, diversion colitis, Behçet's disease, or indeterminate colitis.

In some embodiments, the engineered microorganisms of the present disclosure are administered to a subject to treat a bleed. In some embodiments, the engineered microorganisms of the present disclosure are administered to a subject to treat a gastrointestinal bleed. The terms “treatment,” “treat,” and “treating,” refer to a clinical intervention aimed to reverse, alleviate, delay the onset of, or inhibit the progress of a disease or disorder (e.g., IBD), or one or more symptoms thereof (e.g., gastrointestinal bleeding or inflammation). In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms. Treatment may also be continued after symptoms have resolved, for example, to prevent or delay their recurrence.

Accordingly, also within the scope of the disclosure are pharmaceutical compositions comprising any of the engineered microorganisms disclosed herein. The term “pharmaceutical composition,” as used herein, refers to a composition that can be administrated to a subject in the context of treatment of a disease or disorder (e.g., IBD). In some embodiments, a pharmaceutical composition comprises any of the engineered microorganisms described herein, and a pharmaceutically acceptable excipient. In some embodiments the pharmaceutical compositions are in the form of a pill.

In some embodiments, the methods for detecting bleeding in a subject are disclosed. In some embodiments, methods for detecting bleeding in a subject may include administering any of the engineered microorganisms, described herein, to the subject and obtaining and/or isolating the engineered microorganisms from the subject. For example, from a biological sample (e.g., a stool sample) of the subject. The engineered microorganisms from the subject may be analyzed in vitro to determine if a bleed was detected in the subject. In some embodiments, the engineered microorganisms are analyzed using polymerase chain reaction (PCR), nucleic acid sequencing, measuring the level of an output molecule, or measuring fluorescence from the engineered microorganism. In some embodiments, polymerase chain reaction or nucleic acid sequencing is used to determine whether one or more recombination events occurred within the engineered microorganisms. In some embodiments recombination events indicate the presence of a bleed in the subject. In some embodiments, measuring the level of an output molecule, or the level of fluorescence or luminescence from the microorganism is performed to determine the presence or absence of a bleed in the subject. Analysis of the engineered microorganisms, in some embodiments, is performed to determine the location of a gastrointestinal bleed (e.g. an upper GI bleed or a lower GI bleed). In some embodiments, analysis of the engineered microorganisms is performed to determine the severity of a bleed. In some embodiments, analysis of the engineered microorganisms is performed in vivo. Analysis of the engineered microorganisms in vivo may be performed by measuring fluorescence or luminescence from the engineered microorganisms in the gastrointestinal tract of a subject using methods known in the art, such as endoscopic methods. In some embodiments, one or more results of the analysis of the engineered microorganisms in vivo is transmitted wirelessly (e.g., for real time analysis).

Exemplary HrtR amino acid sequence:
(SEQ ID NO: 1)
MPKSTYFSLSDEKRKRVYDACLLEFQTHSFHEAKIMHIVKALDIPRGSFY
QYFEDLKDSYYYILSQETVEIHDLFFNLLKEYPLEVALNKYKYLLLENLV
NSPQYNLYKYRFLDWTYELERDWKPKGEVTVPARELDNPISQVLKSVIHN
LVYRMFSENWDEQKFIETYDKEIKLLTEGLLNYVTESKK
Exemplary ChuA amino acid sequence:
(SEQ ID NO: 2)
MSRPQFTSLRLSLLALAVSATLPTFAFATETMTVTATGNARSSFEAPMMV
SVIDTSAPENQTATSATDLLRHVPGITLDGTGRTNGQDVNMRGYDHRGVL
VLVDGVRQGTDTGHLNGTFLDPALIKRVEIVRGPSALLYGSGALGGVISY
DTVDAKDLLQEGQSSGFRVFGTGGTGDHSLGLGASAFGRTENLDGIVAWS
SRDRGDLRQSNGETAPNDESINNMLAKGTWQIDSAQSLSGLVRYYNNDAR
EPKNPQTVGASESSNPMVDRSTIQRDAQLSYKLAPQGNDWLNADAKIYWS
EVRINAQNTGSSGEYREQITKGARLENRSTLFADSFASHLLTYGGEYYRQ
EQHPGGATTGFPQAKIDFSSGWLQDEITLRDLPITLLGGTRYDSYRGSSD
GYKDVDADKWSSRAGMTINPTNWLMLFGSYAQAFRAPTMGEMYNDSKHFS
IGRFYTNYWVPNPNLRPETNETQEYGFGLRFDDLMLSNDALEFKASYFDT
KAKDYISTTVDFAAATTMSYNVPNAKIWGWDVMTKYTTDLFSLDVAYNRT
RGKDTDTGEYISSINPDTVTSTLNIPIAHSGFSVGWVGTFADRSTHISSS
YSKQPGYGVNDFYVSYQGQQALKGMTTTLVLGNAFDKEYWSPQGIPQDGR
NGKIFVSYQW
Exemplary HssR amino acid sequence:
(SEQ ID NO: 3)
MVQCLVVDDDPRILNYIASHLQIEHIDAYTQPSGEAALKLLEKQRVDIAV
VDIMMDGMDGFQLCNTLKNDYDIPVIMLTARDALSDKERAFISGTDDYVT
KPFEVKELIFRIRAVLRRYNINSNSEMTIGNLTLNQSYLELQVSNKTMTL
PNKEFQLLFMLAARPKQIFTREQIIEKIWGYDYEGDERTVDVHIKRLRQR
LKKLNATLTIETVRGQGYKVENHV
Exemplary HssS amino acid sequence:
(SEQ ID NO: 4)
MFKTLYARIAIYSITVILFSALISFVLTNVYYHYNLKASNDAKIMKTLKE
ARQYEQSAKPTHIQQYFKHLGQMNYQIMTIDQKGHKTFYGEPFREDTLSQ
NAINNVLNNQDYHGIKDKPFALFVTGFFDNVTDNTVGINFKTKDGSIAVF
MRPDIGETFSEFRTFLAVLLMLLLFISISLVIASTYSIIRPVKKLKLATE
RLIDGDFETPIKQTRKDEIGTLQYHFNKMRESLGQVDQMRQHFVQNVSHE
IKTPLTHIHHLLSELQQTSDKTLRQQYINDIYTITTQLSGLTTELLLLSE
LDNHQHLLFDDKIQVNQLIKDIIRHEQFAADEKSLIILADLESINFLGNQ
RLLHQALSNLLINAIKYTDVGGAIDIALQHSHNNIIFTISNDGSPISPQA
EARLFERFYKVSKHDNSNGLGLAITKSIIELHHGTIQFTQSNEYVTTFTI
TLPNNSL
Exemplary luxCDABE sequences:
LuxC:
(SEQ ID NO: 5)
MTKKISFIINGQVEIFPESDDLVQSINFGDNSVYLPILNDSHVKNIIDCN
GNNELRLHNIVNFLYTVGQRWKNEEYSRRRTYIRDLKKYMGYSEEMAKLE
ANWISMILCSKGGLYDVVENELGSRHIMDEWLPQDESYVRAFPKGKSVHL
LAGNVPLSGIMSILRAILTKNQCIIKTSSTDPFTANALALSFIDVDPNHP
ITRSLSVIYWPHQGDTSLAKEIMRHADVIVAWGGPDAINTWAVEHAPSYA
DVIKFGSKKSLCIIDNPVDLTSAATGAAHDVCFYDQRACFSAQNIYYMGN
HYEEFKLALIEKLNLYAHILPNAKKDFDEKAAYSLVQKESLFAGLKVEVD
IHQRWMIIESNAGVEFNQPLGRCVYLHHVDNIEQILPYVQKNKTQTISIF
PWESSFKYRDALALKGAERIVEAGMNNIFRVGGSHDGMRPLQRLVTYISH
ERPSNYTAKDVAVEIEQTRFLEEDKFLVFVP
LuxD:
(SEQ ID NO: 6)
MENESKYKTIDHVICVEGNKKIHVWETLPEENSPKRKNAIIIASGFARRM
DHFAGLAEYLSRNGFHVIRYDSLHHVGLSSGTIDEFTMSIGKQSLLAVVD
WLTTRKINNFGMLASSLSARIAYASLSEINASFLITAVGVVNLRYSLERA
LGFDYLSLPINELPDNLDFEGHKLGAEVFARDCLDFGWEDLASTINNMMY
LDIPFIAFTANNDNWVKQDEVITLLSNIRSNRCKIYSLLGSSHDLSENLV
VLRNFYQSVTKAAIAMDNDHLDIDVDITEPSFEHLTIATVNERRMRIEIE
NQAISLS
LuxA:
(SEQ ID NO: 7)
MKFGNFLLTYQPPQFSQTEVMKRLVKLGRISEECGFDTVWLLEHHFTEFG
LLGNPYVAAAYLLGATKKLNVGTAAIVLPTAHPVRQLEDVNLLDQMSKGR
FRFGICRGLYNKDFRVFGTDMNNSRALAECWYGLIKNGMTEGYMEADNEH
IKFHKVKVNPAAYSRGGAPVYVVAESASTTEWAAQFGLPMILSWIINTNE
KKAQLELYNEVAQEYGHDIHNIDHCLSYITSVDHDSIKAKEICRKFLGHW
YDSYVNATTIFDDSDQTRGYDFNKGQWRDFVLKGHKDTNRRIDYSYEINP
VGTPQECIDIIQKDIDATGISNICCGFEANGTVDEIIASMKLFQSDVMPF
LKEKQRSLLY
LuxB:
(SEQ ID NO: 8)
MKFGLFFLNFINSTTVQEQSIVRMQEITEYVDKLNFEQILVYENHFSDNG
VVGAPLTVSGFLLGLTEKIKIGSLNHIITTHHPVAIAEEACLLDQLSEGR
FILGFSDCEKKDEMHFFNRPVEYQQQLFEECYEIINDALTTGYCNPDNDF
YSFPKISVNPHAYTPGGPRKYVTATSHHIVEWAAKKGIPLIFKWDDSNDV
RYEYAERYKAVADKYDVDLSEIDHQLMILVNYNEDSNKAKQETRAFISDY
VLEMHPNENFENKLEEIIAENAVGNYTECITAAKLAIEKCGAKSVLLSFE
PMNDLMSQKNVINIVDDNIKKYHMEYT
LuxE:
(SEQ ID NO: 9)
MTSYVDKQEITASSEIDDLIFSSDPLVWSYDEQEKIRKKLVLDAFRNHYK
HCREYRHYCQAHKVDDNITEIDDIPVFPTSVFKFTRLLTSQENEIESWFT
SSGTNGLKSQVARDRLSIERLLGSVSYGMKYVGSWFDHQIELVNLGPDRF
NAHNIWFKYVMSLVELLYPTTFTVTEERIDFVKTLNSLERIKNQGKDLCL
IGSPYFIYLLCHYMKDKKISFSGDKSLYIITGGGWKSYEKESLKRDDFNH
LLFDTFNLSDISQIRDIFNQVELNTCFFEDEMQRKHVPPWVYARALDPET
LKPVPDGTPGLMSYMDASATSYPAFIVTDDVGIISREYGKYPGVLVEILR
RVNTRTQKGCALSLTEAFDS
Exemplary Bxb1 amino acid sequence:
(SEQ ID NO: 10)
MRALVVIRLSRVTDATTSPERQLESCQQLCAQRGWDVVGVAEDLDVSGAV
DPFDRKRRPNLARWLAFEEQPFDVIVAYRVDRLTRSIRHLQQLVHWAEDH
KKLVVSATEAHFDTTTPFAAVVIALMGTVAQMELEAIKERNRSAAHFNIR
AGKYRGSLPPWGYLPTRVDGEWRLVPDPVQRERILEVYHRVVDNHEPLHL
VAHDLNRRGVLSPKDYFAQLQGREPQGREWSATALKRSMISEAMLGYATL
NGKTVRDDDGAPLVRAEPILTREQLEALRAELVKTSRAKPAVSTPSLLLR
VLFCAVCGEPAYKFAGGGRKHPRYRCRSMGFPKHCGNGTVAMAEWDAFCE
EQVLDLLGDAERLEKVWVAGSDSAVELAEVNAELVDLTSLIGSPAYRAGS
PQREALDARIAALAARQEELEGLEARPSGWEWRETGQRFGDWWREQDTAA
KNTWLRSMNVRLTFDVRGGLTRTIDFGDLQEYEQHLRLGSVVERLHTGMS
Exemplary GFPmut3 amino acid sequence:
(SEQ ID NO: 11)
MRKGEELFTGVVPILVELDGDVNGHKFSVSGEGEGDATYGKLTLKFICTT
GKLPVPWPTLVTTFGYGVQCFARYPDHMKQHDFFKSAMPEGYVQERTIFF
KDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHY
LSTQSALSKDPNEKRDHMVLLEFVTAAGITHGMDELYK
Exemplary P_{HrtAB(HrtR)} nucleic acid sequence:
(SEQ ID NO: 12)
TGATGTTAAGAATTTTATTTGTTAGAATTAAATAAATGACACAGTGTCAT
AAATTAAATG
Exemplary P_{HrtAB(HssR)}nucleic acid sequence:
(SEQ ID NO: 13)
GTTCATATTGAGTTCATATTTCAACCTTATACTGACGCTAAAGAAGAAAT
AGGGAGAAGTGAATCGAT
Exemplary P_{L} nucleic acid sequence:
(SEQ ID NO: 14)
TTATCTCTGGCGGTGTTGACATAAATACCACTGGCGGTGATACTGAGCAC
A
Exemplary HrtO nucleic acid sequence:
(SEQ ID NO: 15)
ATGACACAGTGTCAT
Exemplary P_{L(HrtO)} nucleic acid sequence:
(SEQ ID NO: 16)
ATAAATGACACAGTGTCATTTGACAAAATGACACAGTGTCATGATACTGA
GCACA
Exemplary Ribosomal binding site I (RBS1) nucleic
acid sequence:
(SEQ ID NO: 17)
GCTATAAGAAAACACCCTTTATAATCTAGGTTAAT
Exemplary Ribosomal binding site 2 (RBS2) nucleic
acid sequence:
(SEQ ID NO: 18)
ATTAAAGAGGAGAAAG
Exemplary Ribosomal binding site 3 (RBS3) nucleic
acid sequence:
(SEQ ID NO: 19)
TATACTCTAATTAATCACATAATAAGGACGAATTT
Exemplary Ribosomal binding site 4 (RBS4) nucleic
acid sequence:
(SEQ ID NO: 20)
AGCCGCAAACATATAAGGAGGAACCCC
Exemplary P_{HrtAB} nucleic acid sequence:
(SEQ ID NO: 21)
GCTGATTAATATCTGTCAGTAAATTAATTTATCAAAAAACTTAAAAGTA
AAACTACTGACAGGTCTGTCAGTAGTTTTTTTCAATATAAATTCAAATG
ATTGATGTTAAGAATTTTATTTGTTAGAATTAAATAAATGACACAGTGT
CATAAATTAAATG
Exemplary P_{L(tetO)} nucleic acid sequence:
(SEQ ID NO: 22)
TCCCTATCAGTGATAGAGATTGACATCCCTATCAGTGATAGAGATACTG
AGCACATCAGCAGGACGCACTGACC

### EXAMPLES

### Example 1

### Engineered Synthetic Gene Circuits in Escherichia coli for Autonomous in Vivo Sensing of Blood

### Engineered Microorganisms

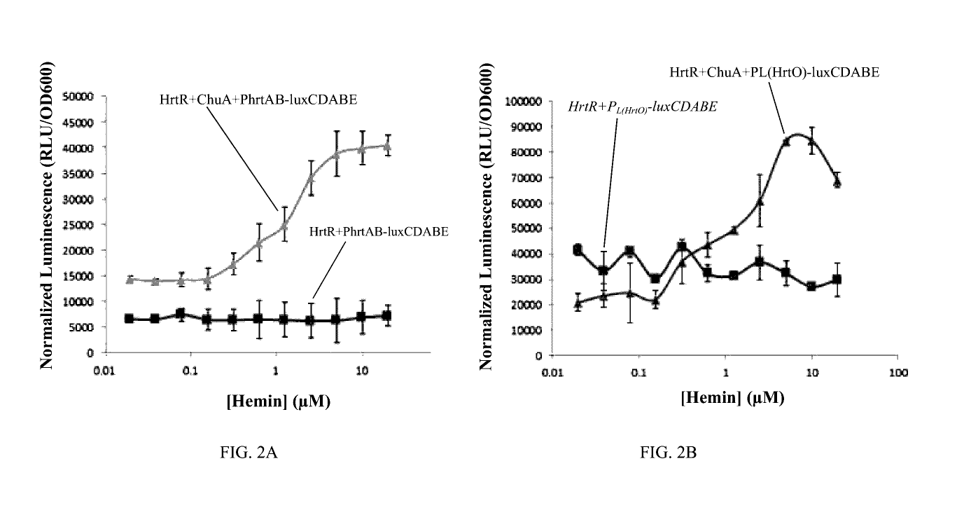

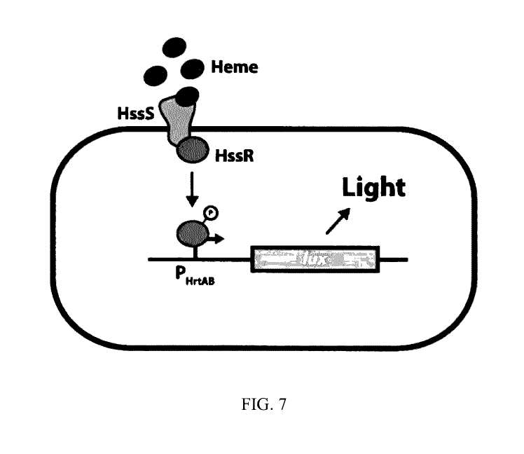

An Escherichia coli MG1655 strain was engineered to sense sub-micromolar levels of hemin using the Lactococcus lactis heme-responsive transcription factor HrtR in conjunction with the E. coli outer-membrane heme transporter ChuA (FIG. 1). Naturally-occurring in Lactococcus lactis, HrtR is a TetR-family transcriptional repressor that tightly binds to cognate HrtO operator sites, thereby preventing transcription of the heme exporter HrtAB. In the presence of heme, HrtR dissociates and permits transcription of HrtAB to protect against heme toxicity. To assess activity of HrtR in E. coli, the heme-responsive promoter P_{hrtAB} was cloned upstream of the bioluminescent reporter luxCDABE. Moreover, a chimeric reporter composed of the strong X, phage promoter P_{L} and the HrtO operator sites (P_{L(HrtO})) was created (FIG. 8). These reporter constructs were independently co-transformed with a plasmid constitutively expressing HrtR into E. coli MG1655. As the reduced form of heme in hemoglobin is rapidly oxidized, the oxidized version, hemin, was used to assay functionality of the heme-responsive genetic circuits. Although the presence of HrtR successfully repressed luminescence from both P_{hrtAB} and P_{L(HrtO)}, the gene circuit was insensitive to the presence of extracellular hemin (FIGS. 2A and 2B). The lack of response may have been due to the fact that the outer membrane of Gram-negative bacteria, such as E. coli, is impermeable to extracellular hemin.

Pathogenic strains of E. coli and other members of Enterobacteriaceae contain a specialized iron-transport system, which imports extracellular heme under iron-starved conditions to be used as a source of cellular iron. Moreover, expression of the outer membrane transporter ChuA is sufficient to allow non-pathogenic K-12 strains of E. coli to import and utilize heme as a sole iron source. By co-expressing ChuA with the HrtR-responsive bioluminescent reporter system, a strain that can luminesce in the presence of hemin was developed (FIGS. 2A and 2B). Both P_{hrtAB} and P_{L(HrtO)} respond to heme in the sub-micromolar range, yielding a 3-4 fold increase in luminescence compared to background. The chimeric promoter provided greater output luminesce than P_{hrtAB}, but also displayed greater background luminescence. Moreover, in initial experiments, the kinetics of the heme-responsive response were characterized and detectable differences were observed in luminescence in 30-60 minutes, depending on the concentration of hemin used (FIGS. 2C and 2D).

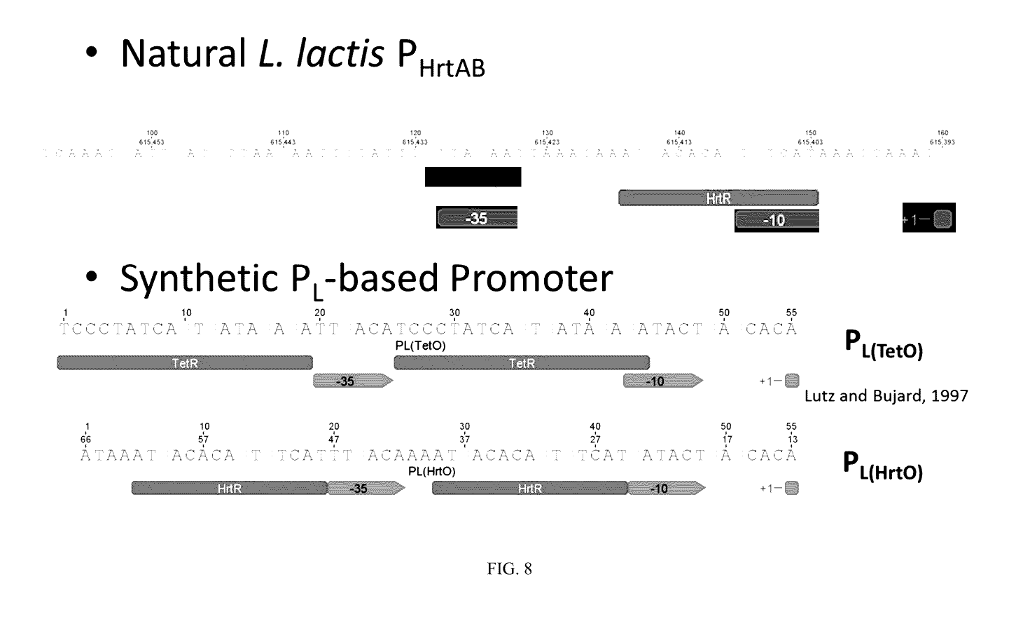

Further characterization of the initial gene circuit, which included P_{L(HrtO)}-luxCDABE and HrtR+ChuA encoded on separate plasmids yields a 4.7 and 5.3 fold increase in luminescence when treated with hemin and horse blood, respectively (FIG. 9A-B). In FIG. 9A-9B, the curves were fit to a Hill function. The Kd refers to the x-axis value at which the y-axis value is half the maximum. Briefly, subcultures were diluted 1:100 in LB with antibiotics and cultured overnight. After 2 hours of initial culture, inducer (hemin or blood) was added as follows: 2-fold dilutions of hemin starting at 20 uM or 3-fold dilutions of blood starting at 0.33%. Samples were read in a plate reader for luminescence and OD600 at 2 hours post-induction. Luminescence values were normalized to OD600. The leftmost point in the graphs of FIG. 9A-B represent uninduced samples.

Due to the low fold change upon heme detection and the high background present in these gene circuits, several strategies to improve performance were explored, including minimizing the number of plasmids, various promoter architectures and improved ribosome binding site strength for HrtR. In the initial design, HrtR and ChuA were encoded on a medium-copy p15a plasmid and the lux operon driven by the synthetic P_{L(hrtO)} promoter was encoded on a high-copy ColE1 plasmid. In order to mitigate the metabolic burden placed by the synthetic gene circuits on host cells and to minimize the chance for plasmid loss in future in vivo models, the HrtR and ChuA gene cassettes were cloned into the same ColE1 plasmid with P_{L(hrtO)}-lux. The resultant construct behaved similarly to the original two-plasmid system. Next, promoter variants which differed in the position of the HrtR operator binding sites with respect to the −35 and -10 sites were constructed. However, the new promoter variants behaved equivalently or worse than the original P_{L(HrtO}) promoter with respect to fold-induction.

### Optimization of HrtR Expression

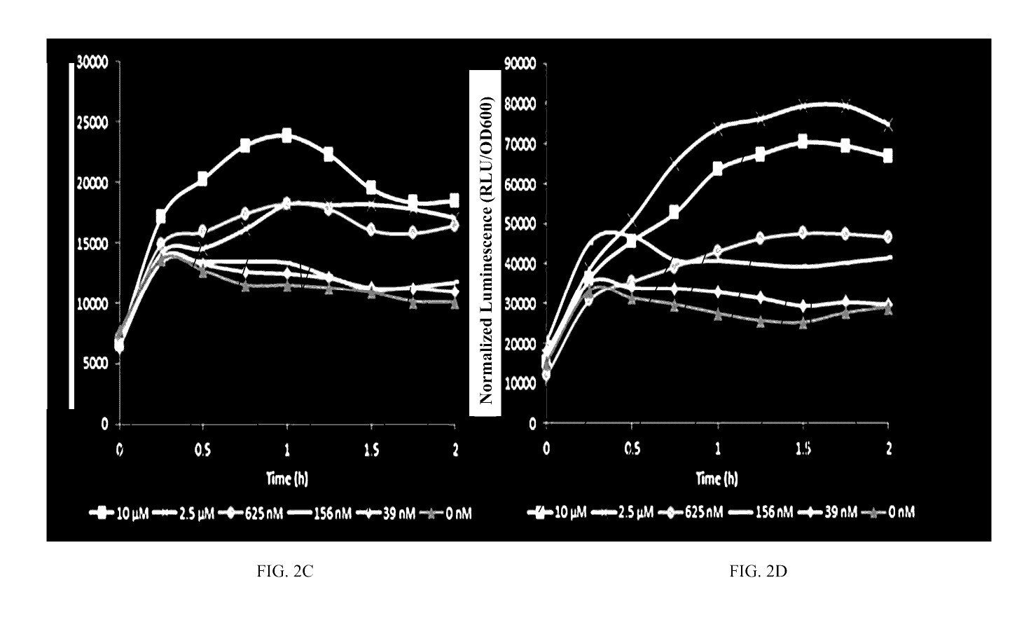

Optimization of the gene circuits provided herein may be performed using a number of approaches including, but not limited to, consolidation of expressed constructs (e.g., HrtR, ChuA, and P_{L(HrtO)}-luxCDABE) into a single plasmid, varied promoter architecture, promoter bashing for increased expression/fold change, and ribosomal binding site (RBS) variation for the ChuA transporter, and/or HrtR repressor. As an initial test, it was determined whether replacing the ribosome binding site (RBS3) of HrtR in the initial gene circuit with a stronger ribosome binding site (RBS2) improved performance of the gene circuit. The improved RBS (RBS2) decreased the baseline expression level by 15-fold and the resultant gene circuit exhibited a 40-60 fold change in luminescence upon induction with blood as compared with RBS3, with detectable changes at <0.01% blood (FIG. 3).

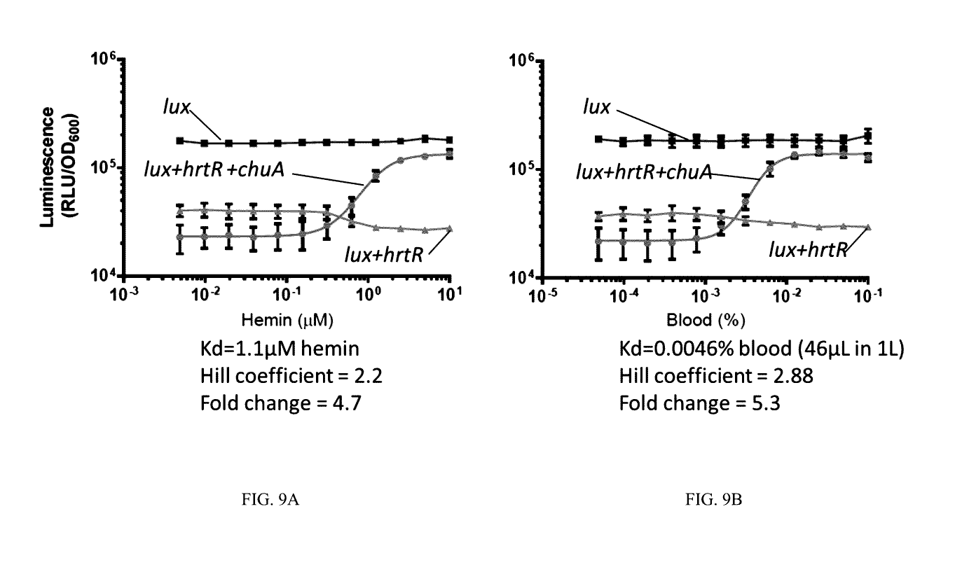

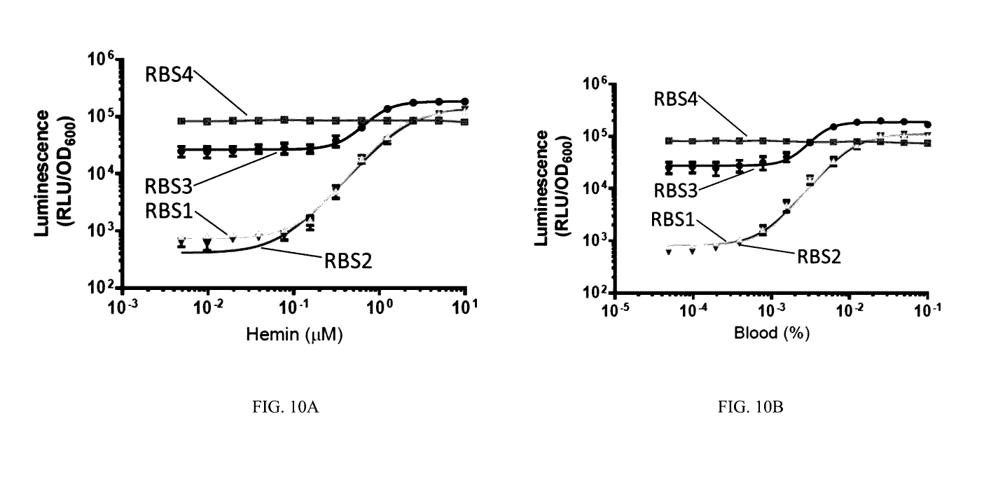

To optimize operation of the gene circuit, expression of HrtR was modulated by engineering various ribosomal binding sequences upstream of HrtR. The predicted strength of the original RBS (RBS3) was determined using the Salis lab RBS calculator and three RBS variants were created based on this value: RBS1, predicted to be 100-fold higher in expression; RBS2 predicted to be 10-fold higher; and RBS4, predicted to be 10-fold lower. DNA encoding four different ribosomal binding sequences, RBS1 (SEQ ID NO: 17), RBS2 (SEQ ID NO: 18), RBS3(SEQ ID NO: 19), and RBS4 (SEQ ID NO: 20) were cloned upstream of HrtR to modulate HrtR expression and the response to increasing concentrations of hemin (FIG. 10A) and horse blood (FIG. 10B) were determined. These experiments were performed using the same protocol as those described in FIGS. 9A and 9B. A summary of the experimental results including predicted strength, Kd, Hill coefficient (Hill) and fold change in luminescence is shown in Table 2. Moreover, the kinetics of response for P_{L(HrtO)}-luxCDABE+HrtR_{RBS2}+ChuA was measured over the course of 2 hours following treatment with hemin (10 μM), blood (0.1%) or an uninduced control (FIG. 11). Overnight cultures were diluted 1:100 in LB+antibiotic and were grown for 2 h prior to addition of inducer. Luminescence and OD₆₀₀ values were measured every 5 minutes until 2 h.

TABLE 2
Gene circuit performance using different RBSs for HrtR.

| | Predicted | | | Fold |
|---|---|---|---|---|
| HrtR RBS | Strength (AU) | Kd | Hill | Change |
| RBS1 | 600 000 | 1.67 μM | 1.9 | 172 |
| RBS2 | 38 000 | 2.11 μM | 1.7 | 341 |
| RBS3 | 3 000 | 0.93 μM | 2.7 | 6 |
| RBS4 | 1 800 | — | — | 0 |

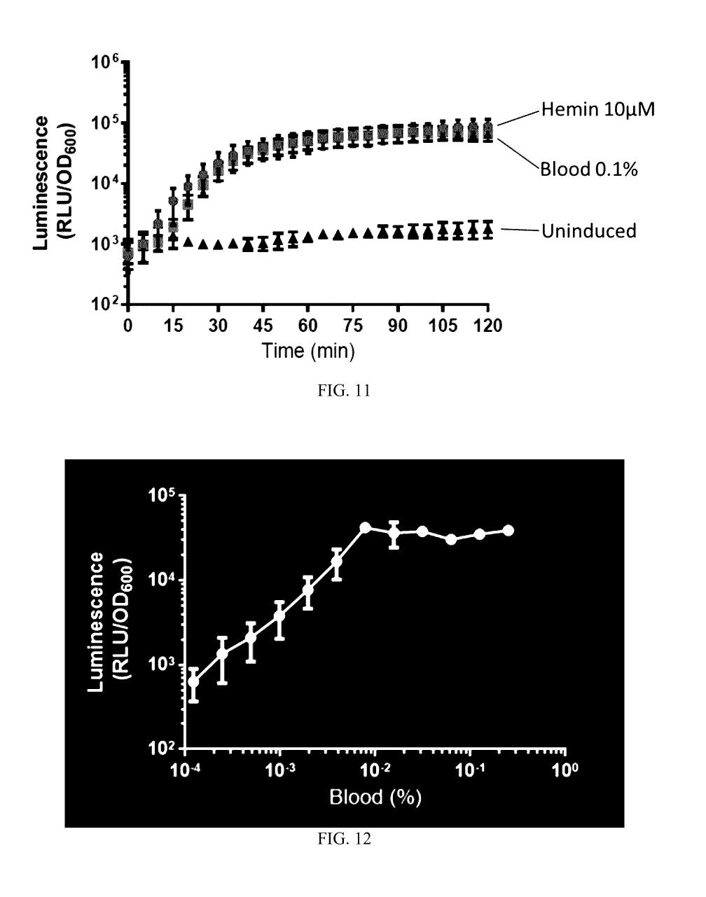

In order to determine whether the genetic circuit used in E. coli MG1655 could be adapted for use in other microorganisms, such as the probiotic bacterium E. coli Nissle 1917, P_{L(HrtO)}-luxCDABE+Hrt_{RBS2}+ChuA was transformed into E. coli Nissle 1917 and tested using the same protocol as FIG. 9 (FIG. 12). The data demonstrate that additional microorganisms, such as probiotic microorganisms, can also be used to detect blood using the constructs and methods provided herein.

As the improved heme-inducible gene circuit performs satisfactorily, the transcriptional circuit can be tested in a rodent model of gastrointestinal bleeding. Bleeding could either be simulated by oral gavage of blood extracted from rats or could be induced using high doses of indomethacin, a non-steroidal anti-inflammatory drug. Using whole animal imaging or intestinal imaging, the inducible luminescence of the engineered strains can be monitored in the presence or absence of gastrointestinal bleeding.

Moreover, cellular memory can be incorporated into the blood-inducible gene circuits. To achieve this, the well-characterized serine recombinase, Bxb1, can be placed under the control of the P_{L(HrtO)} promoter. Expression of Bxb1 in response to extracellular heme will lead to the inversion of a promoter which can drive expression of a reporter gene such as gfp or lux. Design and construction of these recombinase circuits is within the scope of this disclosure.

Detecting and monitoring intestinal bleeding can be both difficult and expensive using existing technologies. However, intestinal bleeding can be serious, and indicative of trauma, cancer, ulcers, varices, and other pathological conditions. As a result, the present disclosure provides, in some aspects, a synthetic-biology platform in a probiotic microbe (e.g., Lactobacillus) to detect gastrointestinal bleeding. The probiotic microbe can be used to detect intestinal bleeding, which is not routinely assayed in situ with current approaches. The present disclosure permits biomarker detection in space- and energy-constrained environments, without requiring major lab equipment or extensive processing. Using synthetic biology, the probiotic microbe was rendered reprogrammable to an array of biomarkers through genetic engineering. Probiotic bacteria can be specially engineered for treatment and diagnostic purposes; to sense disease, report it, and then provide local treatment. With the development of an ingestible intestinal sensor suitable for experimentation in an animal model, this mode of treatment and diagnostics can be developed further. Then, strategies can be employed to achieve tunable input-output transfer functions and digital memory. The probiotic sensors may be assayed to analyze bacteria in the stool or via integrated CMOS sensors that transmit wirelessly. Such technology will permit sensitive and persistent storage of information in DNA for later retrieval.

Existing blood sensors possess significant disadvantages; they do no operate in vivo, are not highly sensitive or specific, and are inconvenient. Endoscopy is invasive and requires specialized training. Fecal immunochemical tests require stool samples and damage or conditions are sensed ex vivo. The PillCam can only detect visual structural damage. FDA-approved blood tests examine blood in the feces (Fecal Occult Blood). The guaiac test is qualitative, as guaiac, a phenol compound present in wood, turns blue in the presence of hemoglobin and hydrogen peroxide. However, dietary peroxidases (vitamin C, etc.) can give a false positive, and large hospital cohort data showed 35% sensitivity in the lower GI and 19% in the upper GI (Lee et al., BMJ Open (2013)). Immunological (anti-hemoglobin) blood tests are quantitative, but hemoglobin is chemically altered as it passes through the digestive tract, changing its antigenicity and reducing the test's sensitivity. It is good for lower GI bleed detection, but not upper GI bleed detection, and large hospital cohort data showed 37% sensitivity in the lower GI (Lee et al., BMJ Open (2013)). Current and proposed methods of upper GI bleed detection are displayed in Table 1.

TABLE 1
Methods of Upper GI Bleed Detection

| Method | Cost | Convenience | Time | Sensitivity |
|---|---|---|---|---|
| Fecal Immunochemical | + | +++ | Short* | − |
| Test | | | | |
| Guaiac Test | + | +++ | Short* | − |
| PillCam | +++ | +++ | Short | + |
| Endoscopy | +++ | + | Long | + |
| Gastric Aspiration | +++ | + | Long | +++ |
| Probiotic Sensor with | + | +++ | Short* | ++ |
| Memory | | | | |
| BacMOS (probiotic | ++ | +++ | Short | ++ |
| integrated with wireless | | | | |
| CMOS) | | | | |

*requires a stool sample for analysis

Common upper gastrointestinal bleeding results in approximately 10 mL of blood loss per day (Rockey et al., Am J Gastro (1999)), which contains approximately 1.5 g hemoglobin. The hemoglobin is diluted in 1 L of material in the stomach, to a concentration of approximately 1.5 g/L, which is approximately 92 μM of heme.

### Lactobacillus Engineering

Lactobacillus is gram-positive, and used for the mucosal delivery of therapeutic molecules or antigens. A well-characterized microbial chassis in synthetic biology E. coli is gram-negative and cannot be easily adapted with sensors and parts form gram-positive bacteria. Lactobactillus has been designated by the FDA as “Generally Recognized As Safe,” or GRAS and is already consumed in a variety of fermented foods. It is safe for human consumption.

Lactobacillus has been engineered to express anti-inflammatory compounds, and engineered bacteria has been used in memory circuits (Gardner et al., 2000; Friedland et al., 2009). E. coli has been used to sense nitric oxide, but not in living animals (Archer et al., 2012). The Lu group recently described the integration of logic and memory devices as well as analog circuits (Nature Biotechnology, 2013; Nature, 2013). Heme sensors have been studied with respect to basic biology, but not for engineered sensors.

### Heme Sensing

The heme sensor system (HssRS) is naturally responsive to sub-μM concentrations of heme, and is found in gram-positive species, such as Straphylococcus aureus, S. epidermidis, Bacillus anthracis and Enterococcus faecalis. HssRS naturally avoids heme toxicity during infection through the upregulation of HrtAB (a heme exporter). Its activation is via a standard two-component system. Heme binds to HssS, a membrane-bound histidine kinase, which autophosphorylates and subsequently phosphorylates the response regulator, HssR. The sensing response is amplified. Then, the phosphorylation of HssR renders it an active transcription factor, and it can mediate activation at the pHrtAB promoter. The number of heme receptors per cell is unknown, but it can be engineered.

Integrating Heme Sensors with Cellular Memory

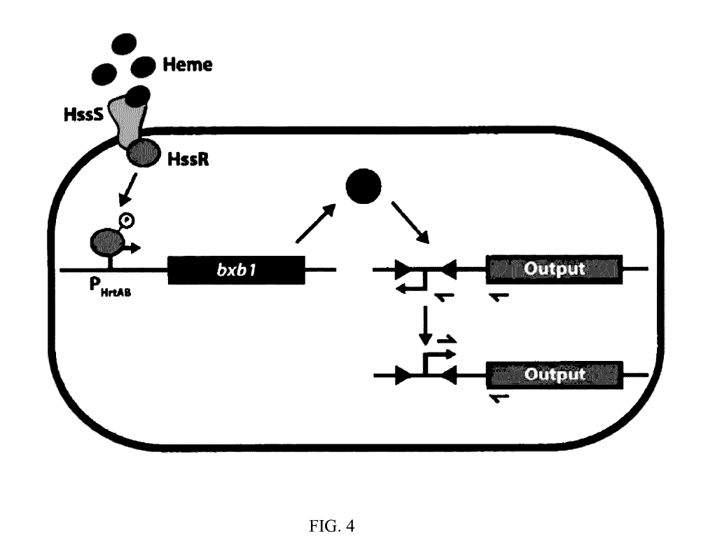

The existing sensor is expected to have approximately 100 nM sensitivity, but circuit topographies can be optimized to tune the transfer functions, through feedback, promoter/RBS engineering, and directed evolution. The heme sensors can be integrated with recombinase-based memory modules to toggle a memory device in the bacteria (FIG. 4). This enables the storage of heme-sensing events in bacterial DNA, which then can be readout at a later time via many different mechanisms, including PCR, sequencing, reporter systems, and other simple assays to query the memory of the cells and readout their respective histories. To date, over 14 orthogonal recombinases for DNA-encoded memory in living cells have been developed (FIG. 5) shows that specific recombinases can be used to invert the orientation of sequences between specific recombinase recognition sites. As shown in FIG. 6, by designing sensors that drive the expression of multiple recombinases, each of which toggles a different memory device, the storage of quantitative sensory information in memory can be achieved. This is useful for multi-parameter pathology assessments in chronic applications.

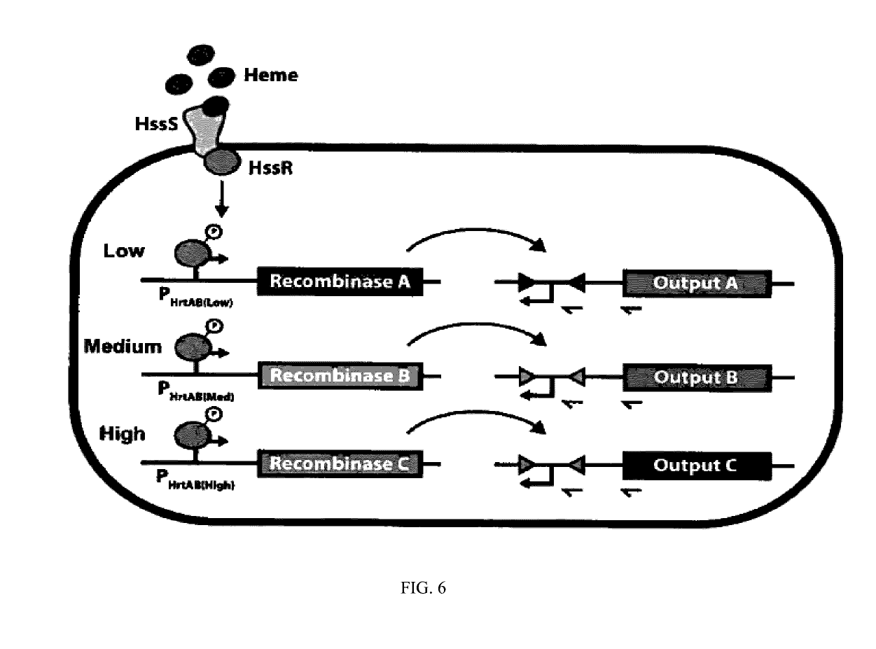

Heme sensors can also be used to drive light expression (FIG. 7), which could be detected in vivo with integrated CMOS sensors with ultra-low-power wireless technology, or ex vivo by detecting light from the engineered microorganisms from a subject.

Lactobacillus has fewer genetic tools than E. coli and there are a limited number of well-characterized biological parts (promoters, RBSes, vectors, secretion tags, etc.). The bioluminescence of L. plantarum may require metabolic engineering, as the FMNH₂ cofactor in the luminescence reaction can be a limiting reagent. Inducible vectors have already been described and modern genomics and synthetic biology techniques enable the rapid engineering of unstudied organisms. Thus, establishing a complementary gram-positive platform for engineered probiotic sensors and therapeutics for in vivo use is within the scope of this disclosure.

### Example 2

### Detection of Bleeding In Vivo

To determine whether the E. coli engineered to sense hemin could be used to detect bleeding in vivo, engineered E. coli transformed with P_{L(HrtO)}-luxCDABE+HrtR_{RBS2}+ChuA were administered to two different mouse models of gastrointestinal (GI) bleeding. Luminescence response of the engineered E. coli was measured using live animal in vivo luminescence imaging, or from measuring luminescence of engineered E. coli obtained from stool.

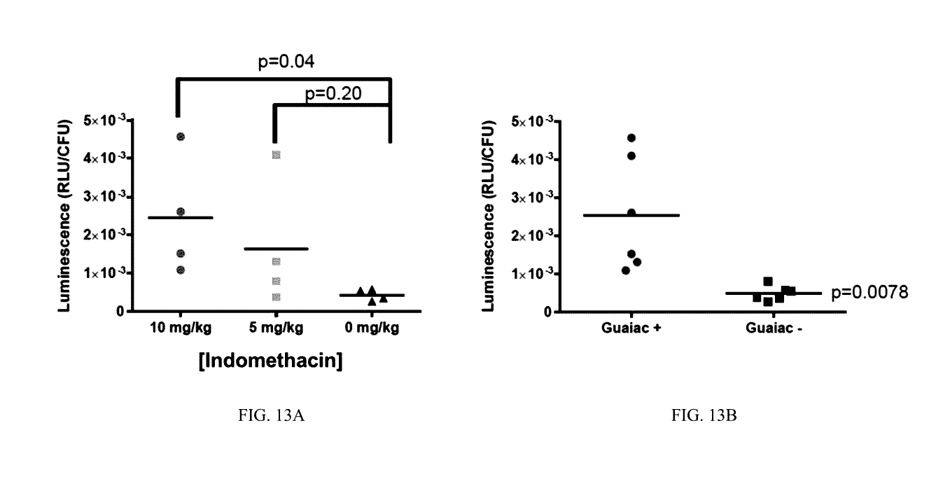

In one example, the nonsteroidal anti-inflammatory drug, indomethacin was used to induce gastric bleeding in mice. Male C57BL/6J mice were gavaged with a single dose of indomethacin, which leads to the formation and hemorrhage of gastric ulcers within several hours of administration. Eighteen hours following the administration of indomethacin, mice were gavaged with engineered E. coli and stool was collected six hours following inoculation. Fecal pellets were homogenized in PBS and subsequently assayed for luminescence, using a plate reader, or for colony counts by serial dilution and plating. The luminescence readout from engineered E. coli obtained from the stool of mice treated with 10 mg/kg indomethacin, 5 mg/kg indomethacin, or vehicle only treated (0 mg/kg indomethacin) mice is shown in FIG. 13A. A statistically significant increase in luminescence was detected from engineered E. coli obtained from mice treated with 10 mg/kg indomethacin. Mice were additionally tested for fecal occult blood using a guaiac test and, when mice are sorted based on the presence of blood in their blood, there was a statistically significant increase in luminescence detected from engineered E. coli that were obtained from mice testing positive for fecal occult blood (FIG. 13B).

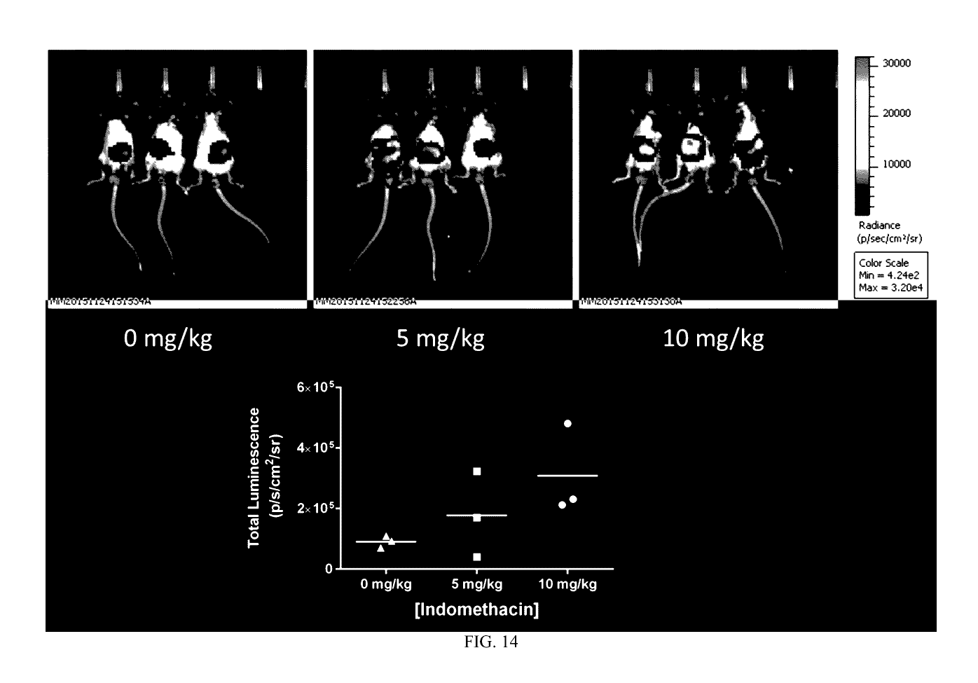

It was also determined whether bleeding could be detected by measuring luminescence of live animals gavaged with E. coli engineered to detect hemin. Male C57BL/6 mice were treated with a single dose of 10 mg/kg of indomethacin, 5 mg/kg of indomethacin, or vehicle only (0 mg/kg of indomethacin). After 18 hours, the mice were gavaged with E. coli engineered to detect hemin. After 6 hours, mice were anesthetized and imaged for luminescence using an IVIS imaging system. Hair was removed from the abdomen of the mice to prevent absorption of light. An increase in luminescence detected from mice, gavaged with engineered E. coli, was observed in mice treated with 5 mg/kg and 10 mg/kg indomethacin as compared to vehicle only treated control mice (0 mg/kg indomethacin). See FIG. 14. The data demonstrate that in vivo bleeding may be detected by direct luminescence imaging of live animals that are administered E. coli engineered to detect hemin.

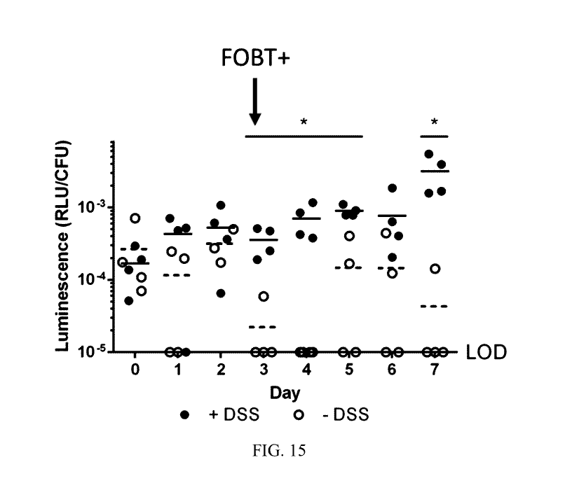

In addition to the NSAID model of gastric bleeding, using indomethacin, it was determined whether E. coli engineered to detect hemin could be used to detect bleeding in a chemically induced colitis mouse model of rectal bleeding. Male C57BL/6J mice were treated with 3% dextran sulfate sodium (DSS) in drinking water beginning on day 0, and were gavaged daily with E. coli transformed with P_{L(HrtO)}-luxCDABE+HrtR_{RBS2}+ChuA. Six hours following gavage of engineered E. coli, stool was collected to measure luminescence and determine colony forming units (CFU) of the engineered E. coli. By day 3 following DSS treatment, fecal occult blood was detected in the stool of mice using a Guaiac test (FIG. 15). A statistically significant increase in luminescence was detected from engineered E. coli obtained from the stool of mice by 3 days following DSS treatment (FIG. 15). These data further demonstrate that in vivo bleeding may be detected by luminescence measurements from engineered E. coli obtained from stool.

### Summary

The present disclosure depicts a new biosensor paradigm, in which, in vivo multiplexed detection of biomarkers is driven by bacterial biosensors. The results may be read out in situ with electronics or in vivo imaging techniques (e.g., in real time), or in the feces via inexpensive nucleic acid technologies. Furthermore, the flexible bacterial-sensing chassis used in biomarker detection are reprogrammable to an array of biomarkers through genetic engineering (synthetic biology). The engineered microorganisms and methods, provided herein, can be useful for detecting inter alia micro-bleeds, inflammation, and cholera, which are not currently assayed in situ. Finally, the biosensing circuit provided herein may be designed for tunable responses.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An engineered microorganism comprising
a heme-responsive transcription factor; and
a genetic circuit responsive to the heme-responsive transcription factor.

2. The engineered microorganism of claim 1 further comprising a heme transporter.

3. The engineered microorganism of claim 1, wherein the heme-responsive transcription factor is a TetR-family transcriptional repressor, and/or wherein the heme responsive transcription factor is from Lactococcus lactis, optionally wherein the heme responsive transcription factor is HrtR.

4.-5. (canceled)

6. The engineered microorganism of claim 1, wherein the microorganism is Escherichia coli or Lactococcus lactis.

7. (canceled)

8. The engineered microorganism of claim 2, wherein the heme transporter is from a gram negative bacterium, and/or wherein the heme transporter is ChuA.

9. (canceled)

10. The engineered microorganism of claim 1, wherein the genetic circuit comprises a first promoter that is operably linked to a nucleic acid sequence encoding an output molecule, wherein the output molecule is a nucleic acid, a reporter polypeptide, a recombinase, or a therapeutic protein, and wherein the first promoter is responsive to the heme-responsive transcription factor.

11. The engineered microorganism of claim 10, wherein the first promoter is a P_{HrtAB(HrtR)} promoter or a P_{L} promoter with one or more HrtO operator sites.

12. The engineered microorganism of claim 1, further comprising a second promoter that is operably linked to a nucleic acid encoding the heme-responsive transcription factor.

13. The engineered microorganism of claim 12, wherein the nucleic acid encoding the heme-responsive transcription factor encodes a ribosomal binding site (RBS), wherein the nucleic acid encoding the RBS comprises the nucleic acid sequence SEQ ID NO: 17, SEQ ID NO: 18, SEQ ID NO: 19, or SEQ ID NO: 20.

14. The engineered microorganism of claim 1, further comprising a third promoter that is operably linked to a nucleic acid encoding the heme transporter.

15.-16. (canceled)

17. The engineered microorganism of claim 1, wherein the heme-responsive transcription factor is a response regulator from a two-component system, optionally wherein the heme responsive transcription factor is HssR.

18. (canceled)

19. The engineered microorganism of claim 17, wherein the engineered microorganism further comprises a heme-responsive histidine kinase from a two-component system, optionally wherein the heme-responsive histidine kinase is HssS.

20. (canceled)

21. The engineered microorganism of claim 19, wherein the genetic circuit comprises
(a) a first promoter that is operably linked to a nucleic acid sequence encoding an output molecule, wherein the first promoter is responsive to the response regulator from a two-component system,
(b) optionally a second promoter that is operably linked to a nucleic acid encoding the response regulator from a two-component system, and
(c) optionally a third promoter that is operably linked to a nucleic acid encoding the heme-responsive histidine kinase from a two-component system.

22.-28. (canceled)

29. The engineered microorganism of claim 1, comprising a first plasmid, wherein the first plasmid encodes the heme-responsive transcription factor, the genetic circuit responsive to the heme-responsive transcription factor, and/or a heme transporter.

30. The engineered microorganism of claim 29, further comprising a second plasmid, wherein the second plasmid encodes the heme-responsive transcription factor, the genetic circuit responsive to the heme-responsive transcription factor, and/or a heme transporter.

31.-33. (canceled)

34. A method of detecting and/or treating bleeding in a subject comprising administering to the subject the engineered microorganism of claim 1.

35.-38. (canceled)

39. The method of claim 34, wherein the subject has, or is at risk of having a bleeding disease or disorder selected from the group consisting of colitis, peptic ulcer disease, liver cirrhosis, inflammatory bowel disease, hemorrhoids, an infection, cancer, a vascular disorder, an adverse effect of a medication, and a blood clotting disorder.

40.-42. (canceled)

43. The method of claim 34, further comprising obtaining the engineered microorganism from the subject and analyzing the engineered microorganism in vitro.

44. (canceled)

45. The method of claim 43, wherein analyzing the engineered microorganism comprises polymerase chain reaction (PCR), nucleic acid sequencing, measuring the level of an output molecule, measuring fluorescence or luminescence from the engineered microorganism, and/or measuring an amount of the engineered microorganism obtained from the subject.

46. The method of claim 34, further comprising analyzing the engineered microorganism in vivo.

47.-49. (canceled)


SEQUENCE LISTING (24 sequences)

SEQ ID NO: 1 - protein, 189 aa, Artificial Sequence
  Synthetic Polypeptide
MPKSTYFSLSDEKRKRVYDACLLEFQTHSFHEAKIMHIVKALDIPRGSFYQYFEDLKDSYYYILSQETVEIHDLFFNLLKEYPLEVALNKYKYLLLENLVNSPQYNLYKYRFLDWTYELERDWKPKGEVTVPARELDNPISQVLKSVIHNLVYRMFSENWDEQKFIETYDKEIKLLTEGLLNYVTESKK

SEQ ID NO: 2 - protein, 660 aa, Artificial Sequence
  Synthetic Polypeptide
MSRPQFTSLRLSLLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVGASESSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 3 - protein, 224 aa, Artificial Sequence
  Synthetic Polypeptide
MVQCLVVDDDPRILNYIASHLQIEHIDAYTQPSGEAALKLLEKQRVDIAVVDIMMDGMDGFQLCNTLKNDYDIPVIMLTARDALSDKERAFISGTDDYVTKPFEVKELIFRIRAVLRRYNINSNSEMTIGNLTLNQSYLELQVSNKTMTLPNKEFQLLFMLAARPKQIFTREQIIEKIWGYDYEGDERTVDVHIKRLRQRLKKLNATLTIETVRGQGYKVENHV

SEQ ID NO: 4 - protein, 457 aa, Artificial Sequence
  Synthetic Polypeptide
MFKTLYARIAIYSITVILFSALISFVLTNVYYHYNLKASNDAKIMKTLKEARQYEQSAKPTHIQQYFKHLGQMNYQIMTIDQKGHKTFYGEPFREDTLSQNAINNVLNNQDYHGIKDKPFALFVTGFFDNVTDNTVGINFKTKDGSIAVFMRPDIGETFSEFRTFLAVLLMLLLFISISLVIASTYSIIRPVKKLKLATERLIDGDFETPIKQTRKDEIGTLQYHFNKMRESLGQVDQMRQHFVQNVSHEIKTPLTHIHHLLSELQQTSDKTLRQQYINDIYTITTQLSGLTTELLLLSELDNHQHLLFDDKIQVNQLIKDIIRHEQFAADEKSLIILADLESINFLGNQRLLHQALSNLLINAIKYTDVGGAIDIALQHSHNNIIFTISNDGSPISPQAEARLFERFYKVSKHDNSNGLGLAITKSIIELHHGTIQFTQSNEYVTTFTITLPNNSL

SEQ ID NO: 5 - protein, 480 aa, Artificial Sequence
  Synthetic Polypeptide
MTKKISFIINGQVEIFPESDDLVQSINFGDNSVYLPILNDSHVKNIIDCNGNNELRLHNIVNFLYTVGQRWKNEEYSRRRTYIRDLKKYMGYSEEMAKLEANWISMILCSKGGLYDVVENELGSRHIMDEWLPQDESYVRAFPKGKSVHLLAGNVPLSGIMSILRAILTKNQCIIKTSSTDPFTANALALSFIDVDPNHPITRSLSVIYWPHQGDTSLAKEIMRHADVIVAWGGPDAINWAVEHAPSYADVIKFGSKKSLCIIDNPVDLTSAATGAAHDVCFYDQRACFSAQNIYYMGNHYEEFKLALIEKLNLYAHILPNAKKDFDEKAAYSLVQKESLFAGLKVEVDIHQRWMIIESNAGVEFNQPLGRCVYLHHVDNIEQILPYVQKNKTQTISIFPWESSFKYRDALALKGAERIVEAGMNNIFRVGGSHDGMRPLQRLVTYISHERPSNYTAKDVAVEIEQTRFLEEDKFLVFVP

SEQ ID NO: 6 - protein, 307 aa, Artificial Sequence
  Synthetic Polypeptide
MENESKYKTIDHVICVEGNKKIHVWETLPEENSPKRKNAIIIASGFARRMDHFAGLAEYLSRNGFHVIRYDSLHHVGLSSGTIDEFTMSIGKQSLLAVVDWLTTRKINNFGMLASSLSARIAYASLSEINASFLITAVGVVNLRYSLERALGFDYLSLPINELPDNLDFEGHKLGAEVFARDCLDFGWEDLASTINNMMYLDIPFIAFTANNDNWVKQDEVITLLSNIRSNRCKIYSLLGSSHDLSENLVVLRNFYQSVTKAAIAMDNDHLDIDVDITEPSFEHLTIATVNERRMRIEIENQAISLS

SEQ ID NO: 7 - protein, 360 aa, Artificial Sequence
  Synthetic Polypeptide
MKFGNFLLTYQPPQFSQTEVMKRLVKLGRISEECGFDTVWLLEHHFTEFGLLGNPYVAAAYLLGATKKLNVGTAAIVLPTAHPVRQLEDVNLLDQMSKGRFRFGICRGLYNKDFRVFGTDMNNSRALAECWYGLIKNGMTEGYMEADNEHIKFHKVKVNPAAYSRGGAPVYVVAESASTTEWAAQFGLPMILSWIINTNEKKAQLELYNEVAQEYGHDIHNIDHCLSYITSVDHDSIKAKEICRKFLGHWYDSYVNATTIFDDSDQTRGYDFNKGQWRDFVLKGHKDTNRRIDYSYEINPVGTPQECIDIIQKDIDATGISNICCGFEANGTVDEIIASMKLFQSDVMPFLKEKQRSLLY

SEQ ID NO: 8 - protein, 327 aa, Artificial Sequence
  Synthetic Polypeptide
MKFGLFFLNFINSTTVQEQSIVRMQEITEYVDKLNFEQILVYENHFSDNGVVGAPLTVSGFLLGLTEKIKIGSLNHIITTHHPVAIAEEACLLDQLSEGRFILGFSDCEKKDEMHFFNRPVEYQQQLFEECYEIINDALTTGYCNPDNDFYSFPKISVNPHAYTPGGPRKYVTATSHHIVEWAAKKGIPLIFKWDDSNDVRYEYAERYKAVADKYDVDLSEIDHQLMILVNYNEDSNKAKQETRAFISDYVLEMHPNENFENKLEEIIAENAVGNYTECITAAKLAIEKCGAKSVLLSFEPMNDLMSQKNVINIVDDNIKKYHMEYT

SEQ ID NO: 9 - protein, 370 aa, Artificial Sequence
  Synthetic Polypeptide
MTSYVDKQEITASSEIDDLIFSSDPLVWSYDEQEKIRKKLVLDAFRNHYKHCREYRHYCQAHKVDDNITEIDDIPVFPTSVFKFTRLLTSQENEIESWFTSSGTNGLKSQVARDRLSIERLLGSVSYGMKYVGSWFDHQIELVNLGPDRFNAHNIWFKYVMSLVELLYPTTFTVTEERIDFVKTLNSLERIKNQGKDLCLIGSPYFIYLLCHYMKDKKISFSGDKSLYIITGGGWKSYEKESLKRDDFNHLLFDTFNLSDISQIRDIFNQVELNTCFFEDEMQRKHVPPWVYARALDPETLKPVPDGTPGLMSYMDASATSYPAFIVTDDVGIISREYGKYPGVLVEILRRVNTRTQKGCALSLTEAFDS

SEQ ID NO: 10 - protein, 500 aa, Artificial Sequence [sequence omitted]

SEQ ID NO: 11 - protein, 238 aa, Artificial Sequence
  Synthetic Polypeptide
MRKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 12 - DNA, 60 bp, Artificial Sequence
  Synthetic Polynucleotide
tgatgttaagaattttatttgttagaattaaataaatgacacagtgtcataaattaaatg

SEQ ID NO: 13 - DNA, 68 bp, Artificial Sequence
  Synthetic Polynucleotide
gttcatattgagttcatatttcaaccttatactgacgctaaagaagaaatagggagaagtgaatcgat

SEQ ID NO: 14 - DNA, 51 bp, Artificial Sequence
  Synthetic Polynucleotide
ttatctctggcggtgttgacataaataccactggcggtgatactgagcaca

SEQ ID NO: 15 - DNA, 15 bp, Artificial Sequence
  Synthetic Polynucleotide
atgacacagtgtcat

SEQ ID NO: 16 - DNA, 55 bp, Artificial Sequence
  Synthetic Polynucleotide
ataaatgacacagtgtcatttgacaaaatgacacagtgtcatgatactgagcaca

SEQ ID NO: 17 - DNA, 35 bp, Artificial Sequence
  Synthetic Polynucleotide
gctataagaaaacaccctttataatctaggttaat

SEQ ID NO: 18 - DNA, 16 bp, Artificial Sequence
  Synthetic Polynucleotide
attaaagaggagaaag

SEQ ID NO: 19 - DNA, 35 bp, Artificial Sequence
  Synthetic Polynucleotide
tatactctaattaatcacataataaggacgaattt

SEQ ID NO: 20 - DNA, 27 bp, Artificial Sequence
  Synthetic Polynucleotide
agccgcaaacatataaggaggaacccc

SEQ ID NO: 21 - DNA, 160 bp, Artificial Sequence
  Synthetic Polynucleotide
gctgattaatatctgtcagtaaattaatttatcaaaaaacttaaaagtaaaactactgacaggtctgtcagtagtttttttcaatataaattcaaatgattgatgttaagaattttatttgttagaattaaataaatgacacagtgtcataaattaaatg

SEQ ID NO: 22 - DNA, 74 bp, Artificial Sequence
  Synthetic Polynucleotide
tccctatcagtgatagagattgacatccctatcagtgatagagatactgagcacatcagcaggacgcactgacc

SEQ ID NO: 23 - DNA, 69 bp, Lactococcus lactis
tcaaatgattgatgttaagaattttatttgttagaattaaataaatgacacagtgtcataaattaaatg

SEQ ID NO: 24 - DNA, 55 bp, Artificial Sequence
  Synthetic Polynucleotide
tccctatcagtgatagagattgacatccctatcagtgatagagatactgagcaca